(19) United States
(12) Patent Application Publication
(10) Pub. No.: US 2023/0016958 A1
(43) Pub. Date: Jan. 19, 2023

(54) CELL CULTURE EVALUATION DEVICE, METHOD FOR OPERATING CELL CULTURE EVALUATION DEVICE, AND PROGRAM FOR OPERATING CELL CULTURE EVALUATION DEVICE

(71) Applicant: FUJIFILM Corporation, Tokyo (JP)

(72) Inventor: Takashi WAKUI, Kanagawa (JP)

(73) Assignee: FUJIFILM Corporation, Tokyo (JP)

(21) Appl. No.: 17/954,145

(22) Filed: Sep. 27, 2022

Related U.S. Application Data
(63) Continuation of application No. PCT/JP2021/005180, filed on Feb. 12, 2021.
(60) Provisional application No. 63/002,696, filed on Mar. 31, 2020.

(51) Int. Cl. G06T 7/00; G06V 10/44

(52) U.S. Cl. CPC G06T 7/0012; G06V 10/44; G06T 2207/20081; G06T 2207/30024; G06T 2207/10056

(57) ABSTRACT

A cell culture evaluation device includes at least one processor. The processor is configured to acquire a cell image obtained by imaging a cell that is being cultured, to input the cell image to an image machine learning model and output an image feature amount set composed of a plurality of types of image feature amounts related to the cell image from the image machine learning model; and to input the image feature amount set to a data machine learning model and output an expression level set composed of expression levels of a plurality of types of ribonucleic acids of the cell from the data machine learning model.

### CROSS-REFERENCE TO RELATED APPLICATIONS

This application is a continuation application of International Application No. PCT/JP2021/005180, filed on Feb. 12, 2021, which is incorporated herein by reference in its entirety. Further, this application claims priority from U.S. Provisional Patent Application No. 63/002,696, filed on Mar. 31, 2020, the disclosure of which is incorporated by reference herein in their entirety.

### BACKGROUND

### Technical Field

The technology of the present disclosure relates to a cell culture evaluation device, a method for operating a cell culture evaluation device, and a non-transitory storage medium storing a program for operating a cell culture evaluation device.

### Related Art

In the field of cell culture, such as induced pluripotent stem (iPS) cell culture, a technique has been proposed which predicts the future quality of a cell in a case in which culture progresses from the present time on the basis of a cell image obtained by imaging the cell at the present time, using a computer. For example, JP2009-044974A discloses a technique that derives a predicted value of the quality of a cell from a plurality of types of index values related to cell morphology.

In the technique disclosed in JP2009-044974A, first, a cell image is input to commercially available image analysis software designed in advance, and a plurality of types of predetermined index values are acquired. Then, the acquired plurality of types of index values are input to a machine learning model using a fuzzy neural network, and the predicted value is output from the machine learning model. Examples of the index value include the area, length, circularity, ellipticity, and radii of the inscribed circle and circumscribed circle of the cell. Examples of the predicted value include a cell proliferation rate, the remaining divisional time, the degree of differentiation, and the degree of canceration. The machine learning model is a model trained with training data which is a combination of a plurality of types of index values acquired from a certain cell image and a measured value of the quality of the cell included in the cell image.

In the field of cell culture, the expression level of a cellular ribonucleic acid (RNA) is important information in predicting the quality of a cell such as the degree of differentiation and the degree of canceration. Currently, a causal relationship between the expression level and the quality of a cell is being studied thoroughly, and it has become clear that the expression level of a specific ribonucleic acid (called a marker) has a great influence on the quality of the cell. However, there are many unexplained parts of the causal relationship between the expression level and the quality of the cell. Therefore, in order to further accelerate the elucidation of the causal relationship between the expression level and the quality of the cell, there is a desire to predict the expression levels of a plurality of types of ribonucleic acids, which are considered to be the basis for predicting the quality of the cell, in addition to the marker.

The following method that imitates the technique disclosed in JP2009-044974A is considered as a method for predicting the expression level. That is, there is a method which inputs a cell image to commercially available image analysis software, acquires a plurality of types of predetermined index values as described above as an example, inputs the acquired plurality of types of index values to a machine learning model, and outputs the expression levels of a plurality of types of ribonucleic acids from the machine learning model. However, the index values are only the values that can be visually and intuitively understood by humans, such as the area, length, and circularity of a cell, and are arbitrarily set by humans. It is considered that these limited index values are unsuitable for the prediction of the expression levels of a plurality of types of ribonucleic acids that have not yet been implemented.

### SUMMARY

The technology of the present disclosure provides a cell culture evaluation device, a method for operating a cell culture evaluation device, and a non-transitory storage medium storing a program for operating a cell culture evaluation device that can appropriately predict expression levels of a plurality of types of ribonucleic acids in a cell.

According to a first aspect of the present disclosure, there is provided a cell culture evaluation device comprising at least one processor. The processor is configured to acquire a cell image obtained by imaging a cell that is being cultured, to input the cell image to an image machine learning model to output an image feature amount set composed of a plurality of types of image feature amounts related to the cell image from the image machine learning model, and to input the image feature amount set to a data machine learning model to output an expression level set composed of expression levels of a plurality of types of ribonucleic acids of the cell from the data machine learning model.

The processor may be configured to perform control to display the expression level set.

The processor may be configured to acquire a plurality of the cell images obtained by imaging one culture container, in which the cell is cultured, a plurality of times, and input each of the plurality of cell images to the image machine learning model to output the image feature amount set for each of the plurality of cell images from the image machine learning model.

The processor may be configured to aggregate a plurality of the image feature amount sets output for each of the plurality of cell images into a predetermined number of image feature amount sets that are capable of being handled by the data machine learning model, and input the aggregated image feature amount sets to the data machine learning model to output the expression level set for each of the aggregated image feature amount sets from the data machine learning model.

The plurality of cell images may include at least one of cell images captured by different imaging methods or cell images obtained by imaging the cells stained with different dyes.

The processor may be configured to input reference information, which is a reference for the output of the expression level set, to the data machine learning model, in addition to the image feature amount set.

The reference information may include morphology-related information of the cell and culture supernatant component information of the cell.

The morphology-related information may include at least one of a type, a donor, a confluency, a quality, or an initialization method of the cell.

In an autoencoder having a compression unit that converts the cell image into the image feature amount set and a restoration unit that generates a restored image of the cell image from the image feature amount set, the compression unit may be used as the image machine learning model.

The compression unit may include: a plurality of extraction units that are prepared according to a size of an extraction target group in the cell image and each of which extracts a target group feature amount set composed of a plurality of types of target group feature amounts for the extraction target group corresponding to the each of the plurality of extraction units, using a convolution layer; and a fully connected unit that converts a plurality of the target group feature amount sets output from the plurality of extraction units into the image feature amount set, using a fully connected layer.

The autoencoder may be trained using a generative adversarial network having a discriminator that determines whether or not the cell image is the same as the restored image.

The autoencoder may be trained by inputting morphology-related information of the cell to the restoration unit, in addition to the image feature amount set from the compression unit.

The morphology-related information may include at least one of a type, a donor, a confluency, a quality, or an initialization method of the cell.

In a convolutional neural network having a compression unit that converts the cell image into the image feature amount set and an output unit that outputs an evaluation label for the cell on the basis of the image feature amount set, the compression unit may be used as the image machine learning model.

According to a second aspect of the present disclosure, there is provided a method for operating a cell culture evaluation device. The method is executed by a processor and comprises: acquiring a cell image obtained by imaging a cell that is being cultured; inputting the cell image to an image machine learning model to output an image feature amount set composed of a plurality of types of image feature amounts related to the cell image from the image machine learning model; and inputting the image feature amount set to a data machine learning model to output an expression level set composed of expression levels of a plurality of types of ribonucleic acids of the cell from the data machine learning model.

According to a third aspect of the present disclosure, there is provided a non-transitory storage medium storing a program that causes a computer to perform a cell culture evaluation processing, the cell culture evaluation processing including: acquiring a cell image obtained by imaging a cell that is being cultured; inputting the cell image to an image machine learning model to output an image feature amount set composed of a plurality of types of image feature amounts related to the cell image from the image machine learning model; and inputting the image feature amount set to a data machine learning model to output an expression level set composed of expression levels of a plurality of types of ribonucleic acids of the cell from the data machine learning model.

According to the technology of the present disclosure, it is possible to provide a cell culture evaluation device, a method for operating a cell culture evaluation device, and a program for operating a cell culture evaluation device that can appropriately predict expression levels of a plurality of types of ribonucleic acids in a cell.

### BRIEF DESCRIPTION OF THE DRAWINGS

FIG. 1 is a diagram illustrating a cell culture evaluation device and the like.

FIG. 2 is a diagram illustrating a plurality of regions obtained by dividing a well.

FIG. 3 is a block diagram illustrating a computer constituting the cell culture evaluation device.

FIG. 4 is a block diagram illustrating a processing unit of a CPU of the cell culture evaluation device.

FIG. 5 is a diagram illustrating a process of a first processing unit.

FIG. 6 is a diagram illustrating a process of an aggregation unit.

FIG. 7 is a diagram illustrating a process of a second processing unit.

FIG. 8 is a diagram illustrating a designation screen.

FIG. 9 is a diagram illustrating a result display screen.

FIG. 10 is a diagram illustrating a configuration of an autoencoder and the formation of an image model.

FIG. 11 is a diagram illustrating an outline of a process in a learning phase of the autoencoder.

FIG. 12 is a diagram illustrating a compression unit.

FIG. 13 is a diagram illustrating an extraction target group in a cell image that each extraction unit of the compression unit is in charge of.

FIG. 14 is a diagram illustrating details of the extraction unit and a fully connected unit.

FIG. 15 is a diagram illustrating details of the extraction unit.

FIG. 16 is a diagram illustrating a convolution process.

FIG. 17 is a diagram illustrating the formation of a target group feature amount map.

FIG. 18 is a diagram in which a convolution process using a filter is applied to the concept of a convolutional neural network.

FIG. 19 is a diagram illustrating a maximum pooling process.

FIG. 20 is a diagram illustrating the concept of a fully connected layer.

FIG. 21 is a diagram illustrating an outline of a process in a learning phase of a data model.

FIG. 22 is a flowchart illustrating a procedure of a process of the cell culture evaluation device.

FIG. 23 is a diagram illustrating an outline of a process in a learning phase of an autoencoder using a generative adversarial network.

FIG. 24 illustrates an aspect in which the autoencoder is incorporated as a generator of the generative adversarial network and a discriminator of the generative adversarial network determines whether or not a training cell image is the same as a training restored image.

FIG. 25A is a diagram illustrating the amount of update of the autoencoder for a determination result of the discriminator and illustrates a case in which the determination result shows that the training cell image is not the same as the training restored image.

FIG. 25B is a diagram illustrating the amount of update of the autoencoder for the determination result of the discriminator and illustrates a case in which the determination result shows that the training cell image is the same as the training restored image.

FIG. 26 is a diagram illustrating a third embodiment in which, in addition to an image feature amount set from the compression unit, morphology-related information of a cell is input to the restoration unit in the learning phase of the autoencoder.

FIG. 27 is a diagram illustrating the morphology-related information.

FIG. 28 is a diagram illustrating a fourth embodiment in which, in addition to the image feature amount set, reference information is input to the data model.

FIG. 29 is a diagram illustrating culture supernatant component information.

FIG. 30 is a diagram illustrating an outline of a process in the learning phase of the data model illustrated in FIG. 28.

FIG. 31 is a diagram illustrating a fifth embodiment in which a compression unit of a convolutional neural network is used as an image model.

FIG. 32 is a diagram illustrating an outline of a process in the learning phase of the convolutional neural network illustrated in FIG. 31.

FIG. 33 is a diagram illustrating an example in which a plurality of cell images are cell images captured by different imaging methods.

FIG. 34 is a diagram illustrating an example in which the plurality of cell images are cell images obtained by imaging cells stained with different dyes.

### DESCRIPTION OF THE PREFERRED EMBODIMENTS

### First Embodiment

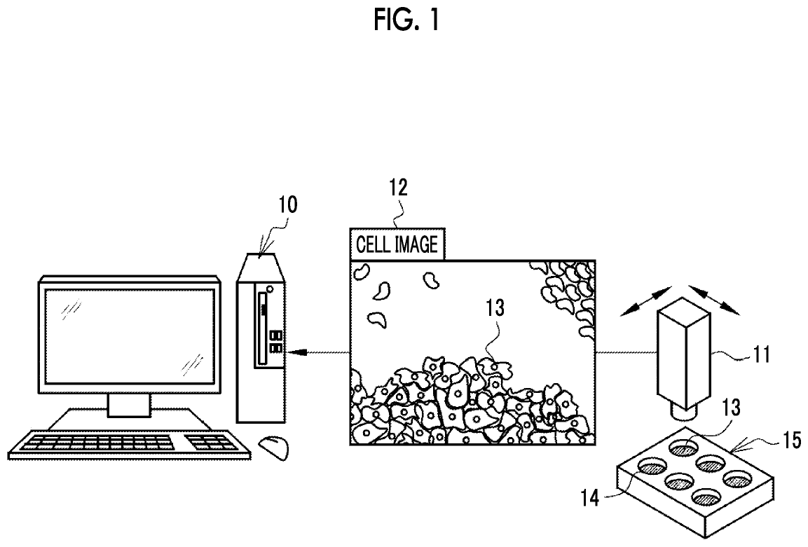

In FIG. 1, a cell culture evaluation device 10 is, for example, a desktop-type personal computer and receives a cell image 12 from an imaging device 11. The imaging device 11 is, for example, a phase-contrast microscope. A well plate 15 in which a plurality of wells 14 for culturing cells 13 are formed is set in the imaging device 11. The imaging device 11 images the cells 13 that are being cultured in one well 14 to obtain the cell image 12. The imaging device 11 transmits the cell image 12 to the cell culture evaluation device 10. The cell 13 is, for example, an iPS cell. In addition, the well 14 is an example of a “culture container” according to the technology of the present disclosure.

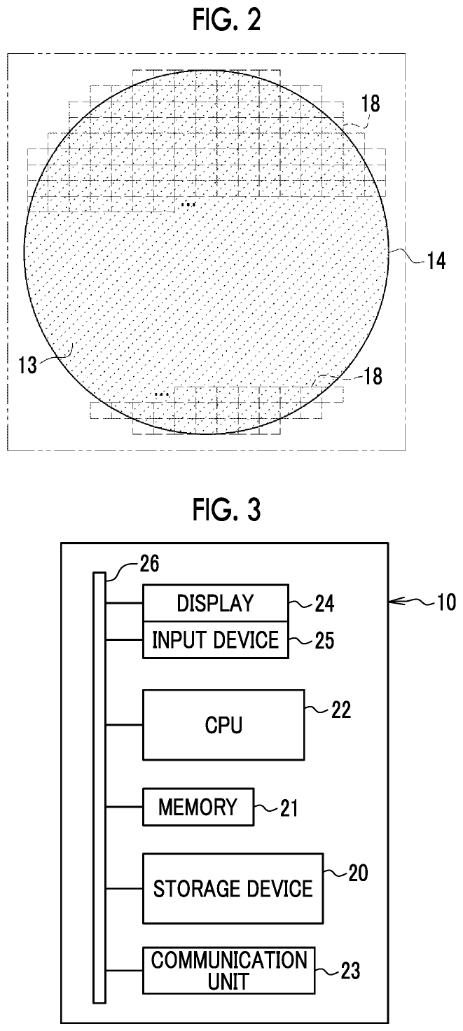

As illustrated in FIG. 2, the imaging device 11 captures a plurality of cell images 12 for each of a plurality of regions 18 obtained by dividing the well 14. Therefore, as represented by arrows in FIG. 1, the imaging device 11 can be moved in two directions orthogonal to each other.

As illustrated in FIG. 3, a computer constituting the cell culture evaluation device 10 comprises a storage device 20, a memory 21, a central processing unit (CPU) 22, a communication unit 23, a display 24, and an input device 25. These are connected to each other through a bus line 26.

The storage device 20 is a hard disk drive that is provided in the computer constituting the cell culture evaluation device 10 or is connected to the computer through a cable or a network. Alternatively, the storage device 20 is a disk array in which a plurality of hard disk drives are connected. The storage device 20 stores, for example, a control program, such as an operating system, various application programs, and various kinds of data associated with these programs. In addition, a solid state drive may be used instead of the hard disk drive.

The memory 21 is a work memory that is used by the CPU 22 to perform processes. The CPU 22 loads a program stored in the storage device 20 to the memory 21 and performs a process corresponding to the program. Therefore, the CPU 22 controls the overall operation of each unit of the computer.

The communication unit 23 controls the transmission of various kinds of information to an external device such as the imaging device 11. The display 24 displays various screens. The computer constituting the cell culture evaluation device 10 receives operation instructions input from the input device 25 through various screens. The input device 25 is, for example, a keyboard, a mouse, or a touch panel.

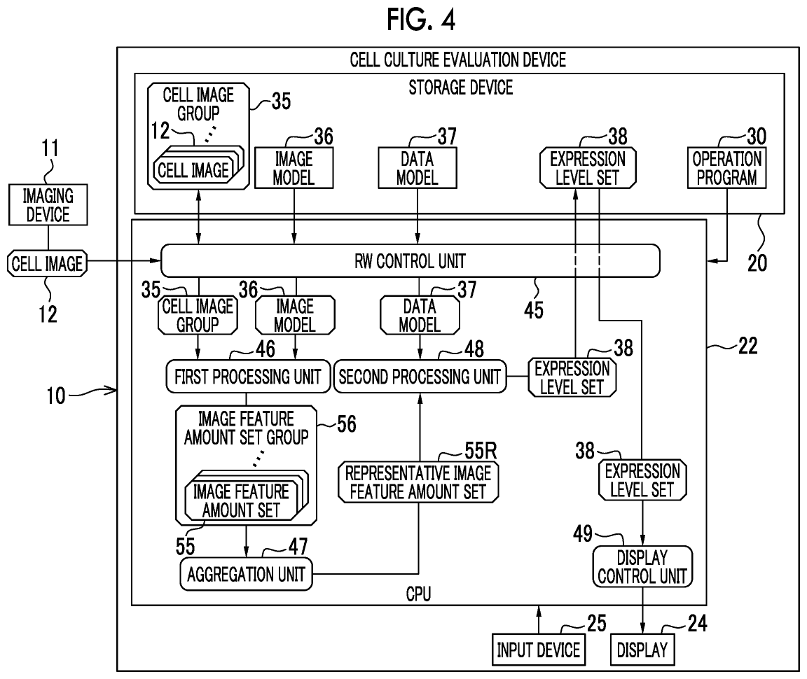

As illustrated in FIG. 4, an operation program 30 is stored in the storage device 20 of the cell culture evaluation device 10. The operation program 30 is an application program for causing the computer to function as the cell culture evaluation device 10. That is, the operation program 30 is an example of a “program for operating a cell culture evaluation device” according to the technology of the present disclosure. The storage device 20 also stores a cell image group 35, an image machine learning model (hereinafter, abbreviated to an image model) 36, a data machine learning model (hereinafter, abbreviated to a data model) 37, and an expression level set 38.

In a case in which the operation program 30 is started, the CPU 22 of the computer constituting the cell culture evaluation device 10 functions as a read and write (hereinafter, abbreviated to RW) control unit 45, a first processing unit 46, an aggregation unit 47, a second processing unit 48, and a display control unit 49 in cooperation with the memory 21 and the like.

The RW control unit 45 controls the storage of various kinds of data in the storage device 20 and the reading of various kinds of data from the storage device 20. For example, the RW control unit 45 receives the cell image 12 from the imaging device 11 and stores the cell image 12 in the storage device 20. Therefore, the cell image group 35 composed of a plurality of cell images 12 obtained by imaging each region 18 of one well 14 is stored in the storage device 20. In addition, only one cell image group 35 is illustrated in FIG. 4. However, a plurality of cell image groups 35 may be stored.

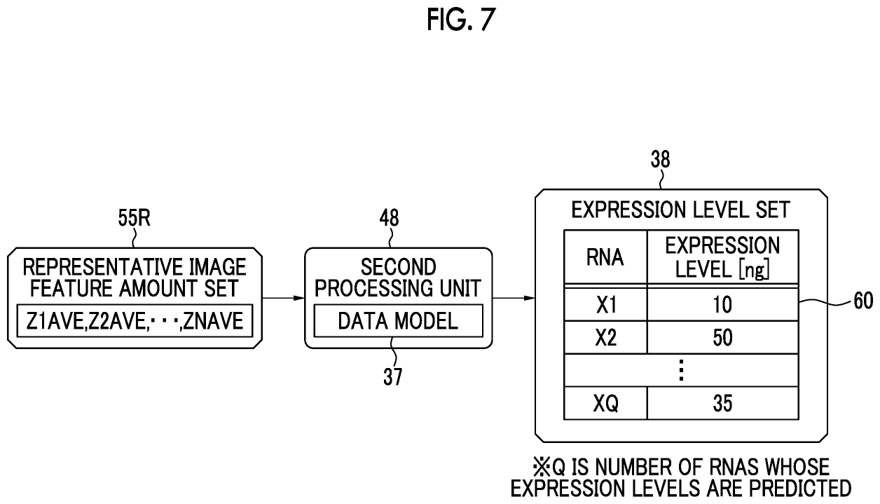

The RW control unit 45 reads the cell image group 35 designated to predict the expression levels of a plurality of types of ribonucleic acids X (see FIG. 7) in the cell 13 from the storage device 20 and outputs the cell image group 35 to the first processing unit 46. The cell image group 35 is composed of a plurality of cell images 12 as described above. Therefore, the RW control unit 45 reads the cell image group 35 from the storage device 20 to acquire the cell images 12. That is, the RW control unit 45 is an example of an “acquisition unit” according to the technology of the present disclosure.

Further, the RW control unit 45 reads the image model 36 from the storage device 20 and outputs the image model 36 to the first processing unit 46. In addition, the RW control unit 45 reads the data model 37 from the storage device 20 and outputs the data model 37 to the second processing unit 48. The data model 37 predicts, for example, the expression level of the ribonucleic acid X on the final day of culture. Furthermore, the data model 37 is a machine learning model, such as a support vector machine, a random forest, or a neural network, and handles only one image feature amount set 55, which will be described below.

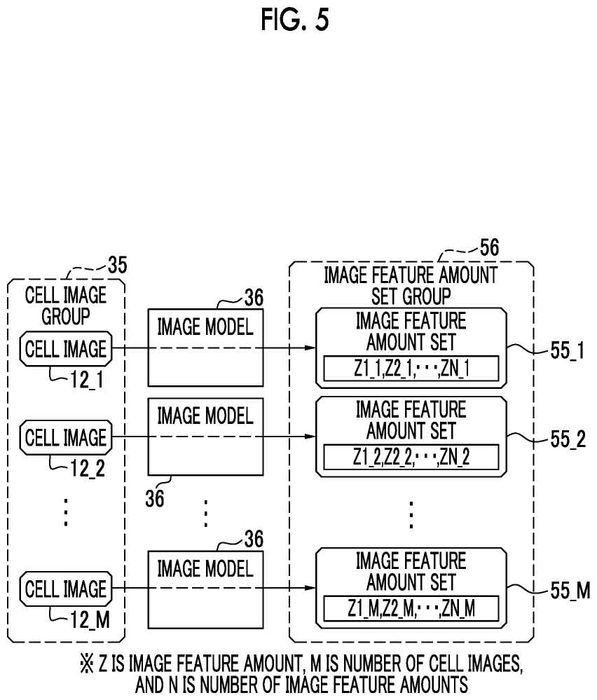

The first processing unit 46 inputs the cell image 12 to the image model 36. Then, the first processing unit 46 outputs an image feature amount set 55 composed of a plurality of types of image feature amounts Z (see FIG. 5) related to the cell image 12 from the image model 36. The first processing unit 46 outputs the image feature amount set 55 for each of the plurality of cell images 12 constituting the cell image group 35. The first processing unit 46 outputs an image feature amount set group 56 composed of a plurality of image feature amount sets 55 corresponding to the plurality of cell images 12 to the aggregation unit 47.

The aggregation unit 47 aggregates the plurality of image feature amount sets 55 constituting the image feature amount set group 56 into one representative image feature amount set 55R that can be handled by the data model 37. The aggregation unit 47 outputs the representative image feature amount set 55R to the second processing unit 48. In addition, the representative image feature amount set 55R is an example of an “aggregated image feature amount set” according to the technology of the present disclosure.

The second processing unit 48 inputs the representative image feature amount set 55R to the data model 37. Then, the second processing unit 48 outputs an expression level set 38 composed of the expression levels of a plurality of types of ribonucleic acids X from the data model 37. The second processing unit 48 outputs the expression level set 38 to the RW control unit 45. In addition, like the cell image group 35, only one expression level set 38 is illustrated in FIG. 4. However, a plurality of expression level sets 38 may be stored.

The RW control unit 45 stores the expression level set 38 in the storage device 20. Further, the RW control unit 45 reads the expression level set 38 from the storage device 20 and outputs the expression level set 38 to the display control unit 49.

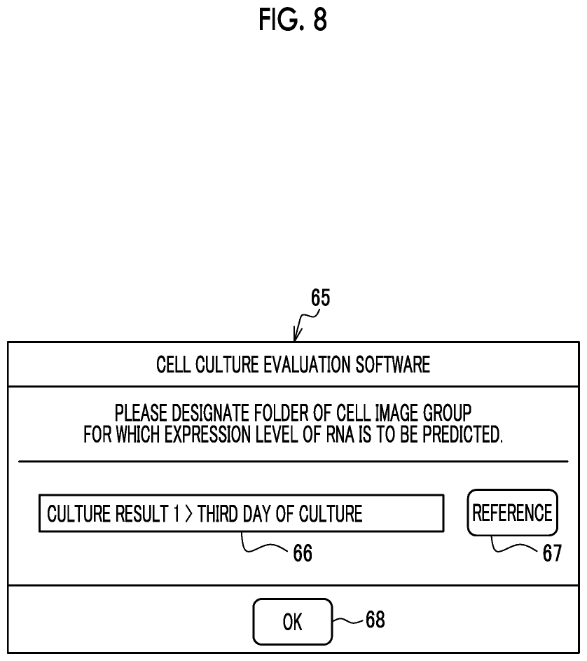

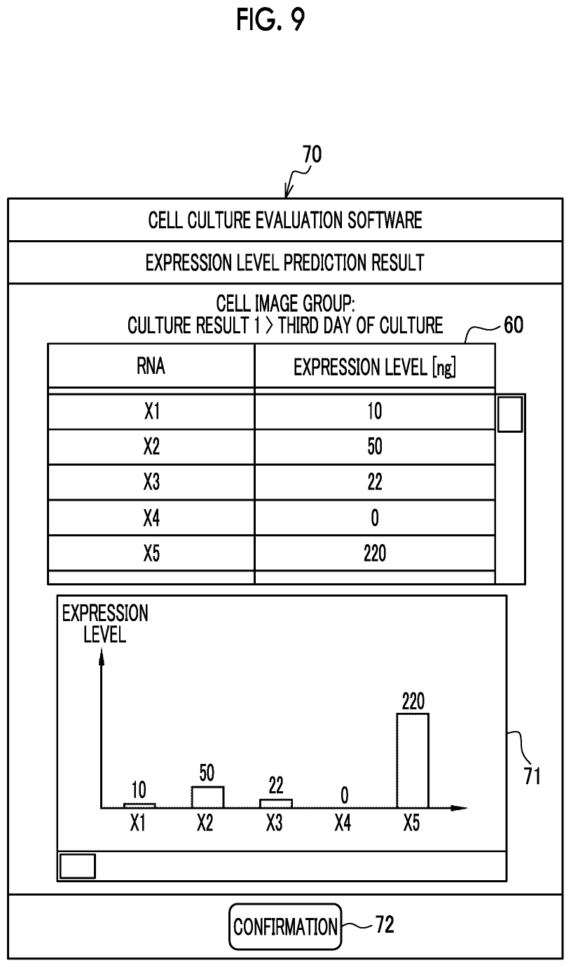

The display control unit 49 controls the display of various screens on the display 24. The various screens include, for example, a designation screen 65 (see FIG. 8) for designating the cell image group 35 for which the expression level is to be predicted and a result display screen 70 (see FIG. 9) for displaying the expression level set 38 as the prediction result of the expression level.

As illustrated in FIG. 5, the first processing unit 46 inputs each of a plurality of cell images 12_1, 12_2, . . . , 12_M constituting the cell image group 35 to the image model 36. Then, the first processing unit 46 outputs image feature amount sets 55_1, 55_2, . . . , 55_M for each of the plurality of cell images 12_1, 12_2, . . . , 12_M from the image model 36. In addition, M is the number of cell images 12 and is, for example, several thousands.

The image feature amount set 55_1 is composed of a plurality of types of image feature amounts Z1_1, Z2_1, . . . , ZN_1. Similarly, the image feature amount set 55_2 is composed of a plurality of types of image feature amounts Z1_2, Z2_2, . . . , ZN_2, and the image feature amount set 55_M is composed of a plurality of types of image feature amounts Z1_M, Z2_M, . . . , ZN_M. In addition, N is the number of image feature amounts and is, for example, several thousands.

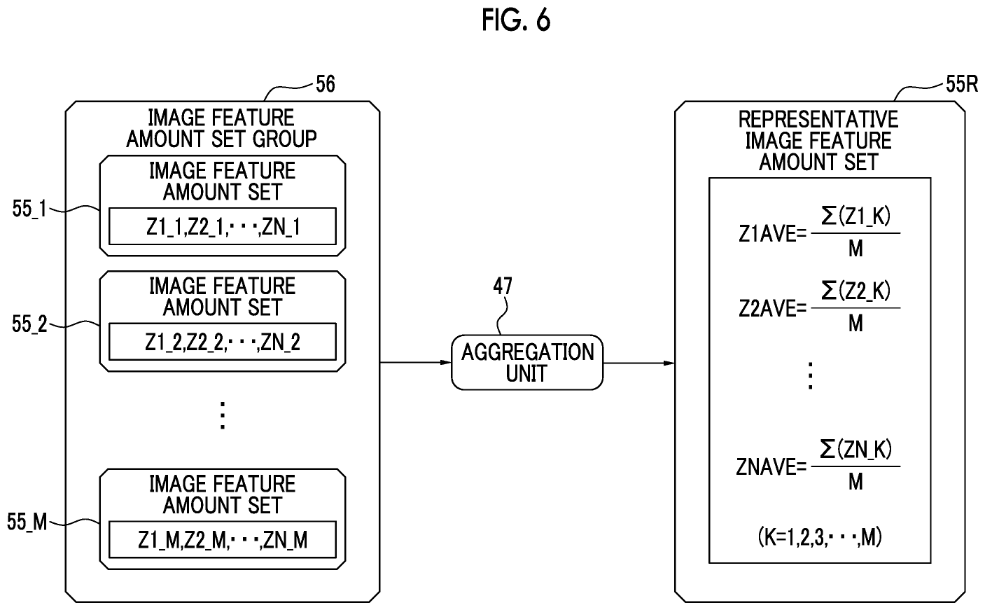

As illustrated in FIG. 6, the aggregation unit 47 calculates average values Z1AVE, Z2AVE, . . . , ZNAVE of the image feature amounts Z1, Z2, . . . , ZN constituting each of the image feature amount sets 55_1, 55_2, . . . , 55_M. For example, the average value Z1AVE of the image feature amounts Z1 is Z1AVE={Σ(Z1_K)}/M (where K=1, 2, 3, . . . , M). The aggregation unit 47 outputs the average values Z1AVE, Z2AVE, ZNAVE calculated in this way as the representative image feature amount set 55R.

As illustrated in FIG. 7, the second processing unit 48 outputs the expression levels of the ribonucleic acids X1, X2, . . . , XQ as the expression level set 38 as represented by a table 60. For example, markers, such as “Nanog”, “Oct4”, “Sox2”, and “Tcf3”, are included in X1, X2, . . . , XQ. In addition, ribonucleic acids other than the markers, for which a causal relationship between the expression level and the quality of the cell 13 has not yet been elucidated, are also included in X1, X2, XQ. The ribonucleic acid X whose expression level is to be predicted is preset by an operator who performs the culture. In addition, Q is the number of ribonucleic acids X whose expression levels are to be predicted and is, for example, 100.

FIG. 8 illustrates the designation screen 65 for designating a cell image group 35 for which the expression level is to be predicted. An input box 66 for directly inputting a folder of the cell image group 35 for which the expression level is to be predicted and a reference button 67 for inputting the folder of the cell image group 35 for which the expression level is to be predicted from Explorer are provided on the designation screen 65. The operator inputs the folder of the cell image group 35 for which the expression level is to be predicted and then selects an OK button 68. Then, the corresponding cell image group 35 is read from the storage device 20 by the RW control unit 45 and is then output to the first processing unit 46.

FIG. 9 illustrates the result display screen 70 for displaying the expression level set 38 as the prediction result of the expression level. The table 60 and a bar graph 71 that show each ribonucleic acid X and the expression level thereof are displayed on the result display screen 70. The result display screen 70 disappears in a case in which a confirmation button 72 is selected.

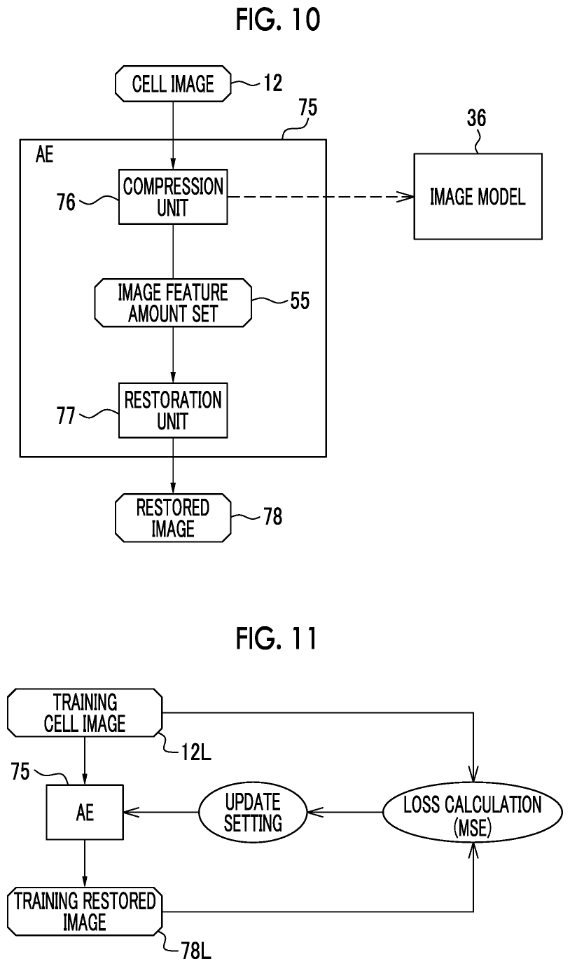

As illustrated in FIG. 10, a compression unit 76 of an autoencoder (hereinafter, abbreviated to AE)) 75 is used as the image model 36. The AE 75 has the compression unit 76 and a restoration unit 77. The cell image 12 is input to the compression unit 76. The compression unit 76 converts the cell image 12 into the image feature amount set 55. The compression unit 76 transmits the image feature amount set 55 to the restoration unit 77. The restoration unit 77 generates a restored image 78 of the cell image 12 from the image feature amount set 55.

As illustrated in FIG. 11, in a learning phase before the compression unit 76 is used as the image model 36, a training cell image 12L is input to the AE 75 to train the AE 75. The AE 75 outputs a training restored image 78L for the training cell image 12L. The AE 75 performs loss calculation on the basis of the training cell image 12L and the training restored images 78L, using a loss function such as a mean squared error (MSE). Then, the update setting of various coefficients of the AE 75 is performed according to the result of the loss calculation, and the AE 75 is updated according to the update setting.

In the learning phase of the AE 75, the above-described series of processes of the input of the training cell image 12L to the AE 75, the output of the training restored image 78L from the AE 75, the loss calculation, the update setting, and the update of the AE 75 are repeatedly performed while the training cell image 12L is being exchanged. The repetition of the series of processes ends in a case in which the accuracy of restoration from the training cell image 12L to the training restored image 78L reaches a predetermined set level. The compression unit 76 of the AE 75 whose restoration accuracy has reached the set level in this way is stored in the storage device 20 and used as the image model 36.

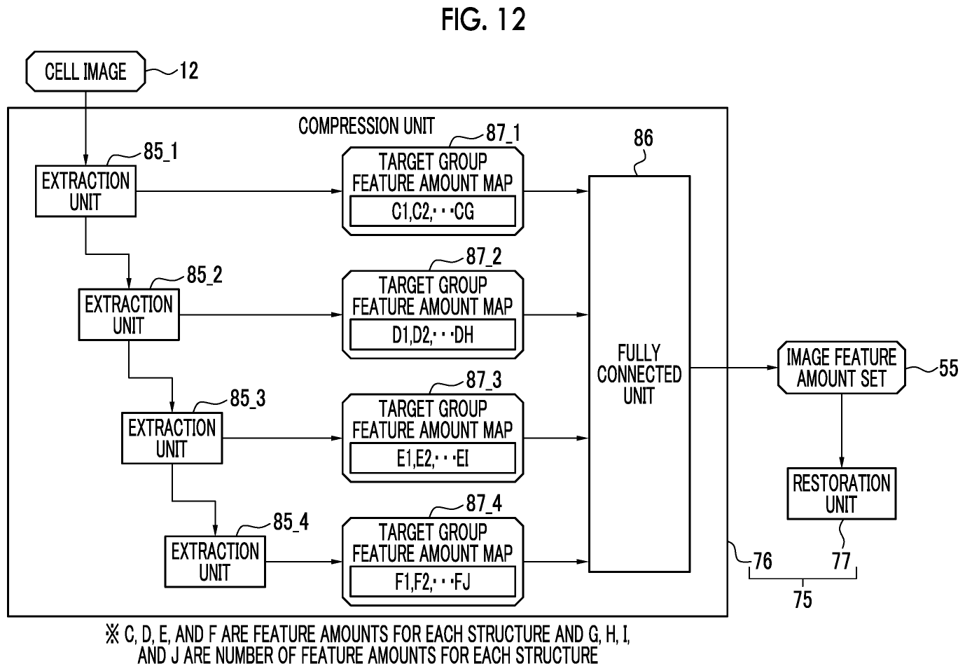

As illustrated in FIG. 12, the compression unit 76 has four extraction units 85_1, 85_2, 85_3, and 85_4 and a fully connected unit 86. The extraction units 85_1, 85_2, 85_3, and 85_4 extract target group feature amount maps 87_1, 87_2, 87_3, and 87_4, respectively. The extraction units 85_1, 85_2, 85_3, and 85_4 output the target group feature amount maps 87_1, 87_2, 87_3, and 87_4 to the fully connected unit 86. The fully connected unit 86 converts the target group feature amount maps 87_1, 87_2, 87_3, and 87_4 into the image feature amount set 55. The target group feature amount maps 87_1, 87_2, 87_3, 87_4 are examples of a “target group feature amount set” according to the technology of the present disclosure. In addition, in the following description, in a case in which the extraction units 85_1, 85_2, 85_3, and 85_4 do not need to be particularly distinguished from each other, they may be collectively referred to as extraction units 85. Similarly, the target group feature amount maps 87_1, 87_2, 87_3, and 87_4 may be collectively referred to as target group feature amount maps 87.

The target group feature amount map 87_1 is composed of a plurality of types of target group feature amounts C1, C2, . . . , and CG Similarly, the target group feature amount map 87_2 is composed of a plurality of types of target group feature amounts D1, D2, . . . , DH, and the target group feature amount map 87_3 is composed of a plurality of types of target group feature amounts E1, E2, . . . , EI. The target group feature amount map 87_4 is composed of a plurality of types of target group feature amounts F1, F2, . . . , FJ. In addition, G, H, I, and J are the numbers of target group feature amounts C, D, E, and F, respectively, and are, for example, tens of thousands to hundreds of thousands.

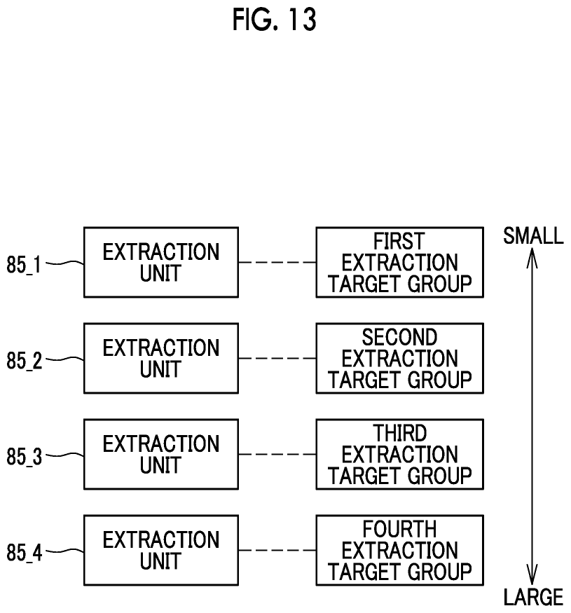

As illustrated in FIG. 13, the extraction unit 85_1 is for a first extraction target group among the objects in the cell image 12. Similarly, the extraction unit 85_2 is for a second extraction target group, the extraction unit 85_3 is for a third extraction target group, and the extraction unit 85_4 is for a fourth extraction target group.

The first extraction target group that the extraction unit 85_1 is in charge of is an extraction target group having the smallest size among the extraction target groups. Therefore, the target group feature amount map 87_1 extracted from the extraction unit 85_1 indicates the features of an extraction target group having a relatively small size among the extraction target groups in the cell image 12. On the other hand, the fourth extraction target group that the extraction unit 85_4 is in charge of is an extraction target group having the largest size among the extraction target groups. Therefore, the target group feature amount map 87_4 extracted from the extraction unit 85_4 indicates the features of an extraction target group having a relatively large size among the extraction target groups in the cell image 12.

The second extraction target group that the extraction unit 85_2 is in charge of and the third extraction target group that the extraction unit 85_3 is in charge of are extraction target groups having a medium size between the first extraction target group and the fourth extraction target group. Therefore, the target group feature amount maps 87_2 and 87_3 respectively extracted from the extraction units 85_2 and 85_3 indicate the features of extraction target groups having a medium size between the extraction target group having a small size and the extraction target group having a large size.

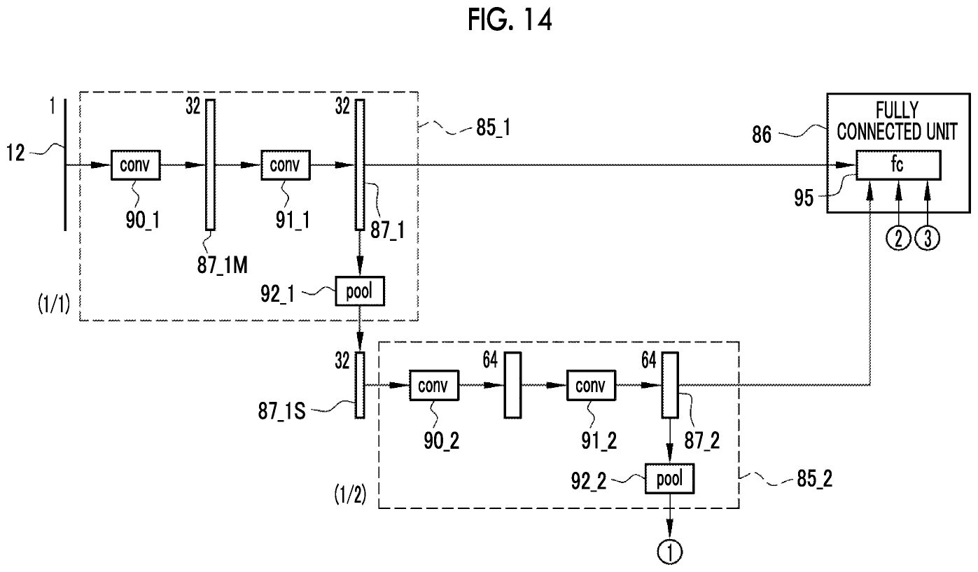

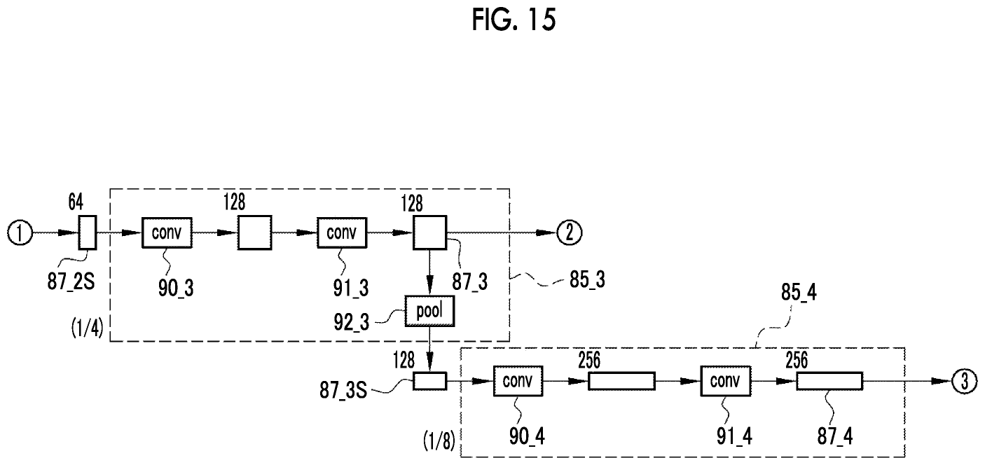

As illustrated in FIGS. 14 and 15, the extraction unit 85 has two convolution layers 90 and 91. Further, the extraction unit 85 other than the extraction unit 85_4 has a pooling layer 92. The fully connected unit 86 has a fully connected layer 95. The convolution layers 90 and 91 are represented by “cony” (abbreviation of “convolution”), and the pooling layer 92 is represented by “pool (abbreviation of pooling)”. Further, the fully connected layer 95 is represented by “fc (abbreviation of fully connected)”. In addition, an activation function and batch normalization are introduced into the extraction unit 85, which is not illustrated.

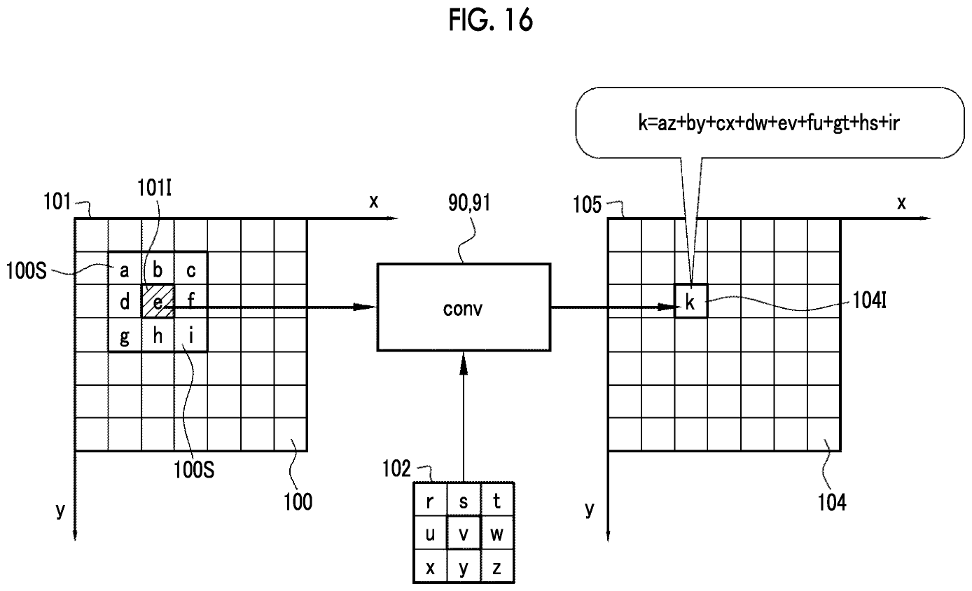

The convolution layers 90 and 91 perform, for example, a convolution process illustrated in FIG. 16. That is, a 3×3 filter 102 is applied to input data 101 having a plurality of pixels 100 which are two-dimensionally arranged. Then, a pixel value e of one pixel of interest 1001 among the pixels 100 and pixel values a, b, c, d, f, g, h, and i of eight pixels 100S adjacent to the pixel of interest 1001 are convoluted. Therefore, output data 105 having a plurality of pixels 104 which are two-dimensionally arranged is obtained similarly to the input data 101. That is, the pixel values of the pixels 104 of the output data 105 are the target group feature amounts C, D, E, and F.

In a case in which coefficients of the filter 102 are r, s, t, u, v, w, x, y, and z, a pixel value k of a pixel of interest 1041 of the output data 105, which is the result of the convolution operation on the pixel of interest 1001, is obtained by calculating, for example, the following Expression (1).

k=az+by+cx+dw+ev+fu+gt+hs+ir (1)

In the convolution process, the above-mentioned convolution operation is sequentially performed on each pixel 100 of the input data 101, and the pixel value of the pixel 104 of the output data 105 is output. In this way, the output data 105 in which the pixel values of the pixels 100 of the input data 101 have been convoluted is output.

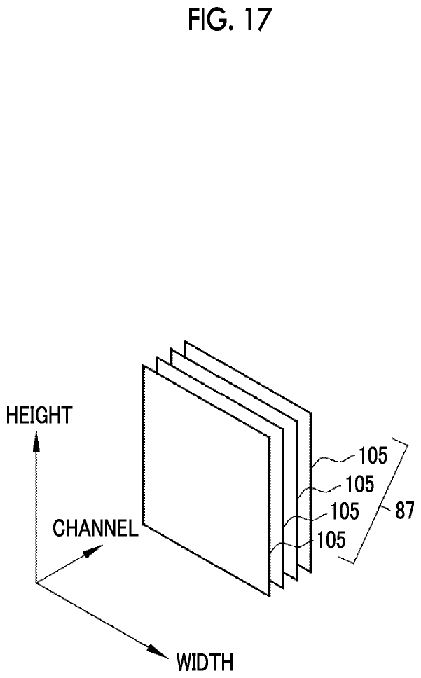

One output data item 105 is output for one filter 102. In a case in which a plurality of types of filters 102 are applied to one input data item 101, the output data 105 is output for each filter 102. That is, as illustrated in FIG. 17, the number of output data items 105 is equal to the number of filters 102 applied to the input data 101. Further, since the output data 105 has a plurality of pixels 104 which are two-dimensionally arranged, it has a width and a height. In a case in which a plurality of output data items 105 are output, the target group feature amount map 87 is a set of the plurality of output data items 105. In the target group feature amount map 87, the number of output data items 105 is called the number of channels. FIG. 17 illustrates a four-channel target group feature amount map 87 which has four output data items 105 output by applying four filters 102 to the input data 101.

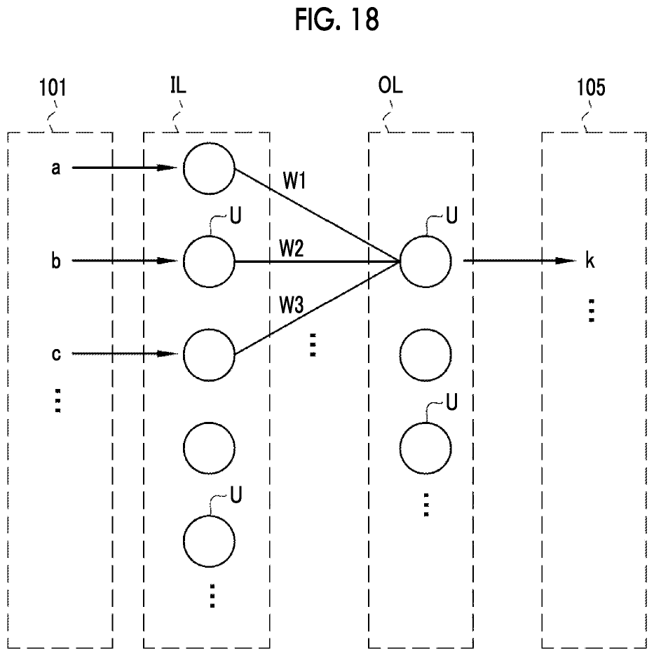

FIG. 18 is a diagram in which a convolution process using the filter 102 is applied to the concept of a convolutional neural network. First, it is assumed that the convolutional neural network has an input layer IL and an output layer OL which have a plurality of units U. In this case, weights W1, W2, W3, . . . indicating the strength of the connection between each unit U of the input layer IL and each unit U of the output layer OL correspond to the coefficients r to z of the filter 102. Pixel values a, b, c, . . . of each pixel 100 of the input data 101 are input to each unit U of the input layer IL. The sum of the products of the pixel values a, b, c, and the weights W1, W2, W3, . . . is an output value from each unit U of the output layer OL. This output value corresponds to the pixel value k of the output data 105. In the learning phase of the AE 75 illustrated in FIG. 11, the coefficients r to z of the filter 102 corresponding to the weights W1, W2, W3, . . . are updated.

The pooling layer 92 performs a pooling process on the target group feature amount map 87. The pooling process is a process that calculates the local statistics of the target group feature amount map 87 and reduces the size (width×height) of the target group feature amount map 87 such that the target group feature amount map 87 is a reduction target group feature amount map 87S.

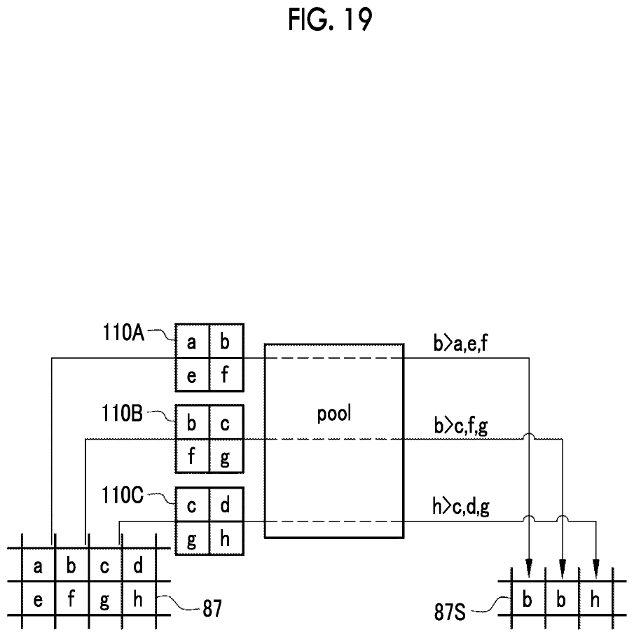

As the pooling process, for example, a maximum pooling process illustrated in FIG. 19 is performed. In FIG. 19, the maximum pooling process is, for example, a process that calculates the maximum value of the pixel values in a block 110 of 2×2 pixels in the target group feature amount map 87 as a local statistic and sets the calculated maximum value as the pixel value of the pixel in the reduction target group feature amount map 87S. In a case in which the maximum pooling process is performed while shifting the block 110 by one pixel in a width direction and a height direction, the reduction target group feature amount map 87S is reduced to half the size of the original target group feature amount map 87.

FIG. 19 illustrates a case in which b among pixel values a, b, e, and fin a block 110A, b among pixel values b, c, f, and g in a block 110B, and h among pixel values c, d, g, and h in a block 110C are the maximum. In addition, an average pooling process may be performed which calculates an average value as the local statistic instead of the maximum value.

In FIGS. 14 and 15, for example, numbers 32 and 64 on the target group feature amount maps 87 indicate the number of output data items 105 in each target group feature amount map 87, that is, the number of channels. The numbers in parentheses, such as 1/1 and 1/2, written on the lower left side of each of the extraction units 85_1, 85_2, 85_3, and 85_4 indicate the size of the input data 101 handled by each of the extraction units 85_1, 85_2, 85_3, and 85_4 based on the size of the cell image 12.

In FIG. 14, the cell image 12 is input as the input data 101 to the extraction unit 85_1, and each of the two convolution layers 90_1 and 91_1 performs the convolution process on the cell image 12. First, the convolution layer 90_1 performs a convolution process of applying 32 filters 102 on the cell image 12 to extract a 32-channel intermediate target group feature amount map 87_1M. Then, the convolution layer 91_1 further performs the convolution process of applying 32 filters 102 on the intermediate target group feature amount map 87_1M. Finally, a 32-channel target group feature amount map 87_1 is extracted. The target group feature amount map 87_1 is output to the fully connected layer 95 of the fully connected unit 86.

The size of the target group feature amount map 87_1 finally extracted by the extraction unit 85_1 is the same as the size of the cell image 12. Therefore, the size handled by the extraction unit 85_1 is the same as that of the cell image 12, that is, 1/1 indicating the same magnification.

The pooling layer 92_1 performs the maximum pooling process on the target group feature amount map 87_1 such that the target group feature amount map 87_1 is a half-size reduction target group feature amount map 87_1S. The pooling layer 92_1 outputs the reduction target group feature amount map 87_1S to the extraction unit 85_2. That is, the reduction target group feature amount map 87_1S which has been reduced to half the size of the cell image 12 is input as the input data 101 to the extraction unit 85_2.

In the extraction unit 85_2, the convolution layers 90_2 and 91_2 perform a convolution process of applying 64 filters 102 twice on the reduction target group feature amount map 87_1S from the extraction unit 85_1. Finally, a 64-channel target group feature amount map 87_2 is extracted. The target group feature amount map 87_2 is output to the fully connected layer 95 of the fully connected unit 86.

The pooling layer 92_2 performs the maximum pooling process on the target group feature amount map 87_2 such that the target group feature amount map 87_2 is a half-size reduction target group feature amount map 87_2S (see FIG. 15). The pooling layer 92_2 outputs the reduction target group feature amount map 87_2S to the extraction unit 85_3. That is, the reduction target group feature amount map 87_2S which has been reduced to ¼ of the size of the cell image 12 is input as the input data 101 to the extraction unit 85_3.

In FIG. 15, in the extraction unit 85_3, the convolution layers 90_3 and 91_3 perform a convolution process of applying 128 filters 102 twice on the reduction target group feature amount map 87_2S from the extraction unit 85_2. Finally, a 128-channel target group feature amount map 87_3 is extracted. The target group feature amount map 87_3 is output to the fully connected layer 95 of the fully connected unit 86.

The pooling layer 92_3 performs the maximum pooling process on the target group feature amount map 87_3 such that the target group feature amount map 87_3 is a half-size reduction target group feature amount map 87_3S. The pooling layer 92_3 outputs the reduction target group feature amount map 87_3S to the extraction unit 85_4. That is, the reduction target group feature amount map 87_3S which has been reduced to ⅛ of the size of the cell image 12 is input as the input data 101 to the extraction unit 85_4.

In the extraction unit 85_4, the convolution layers 90_4 and 91_4 perform a convolution process of applying 256 filters 102 twice on the reduction target group feature amount map 87_3S from the extraction unit 85_3. Finally, a 256-channel target group feature amount map 87_4 is extracted. The target group feature amount map 87_4 is output to the fully connected layer 95 of the fully connected unit 86.

As described above, the input data 101 (the cell image 12 or the reduction target group feature amount map 87S) input to each of the extraction units 85_1, 85_2, 85_3, and 85_4 is gradually reduced in size and resolution from the highest extraction unit 85_1 to the lowest extraction unit 85_4. In this example, the input data 101 having a size that is 1/1 (equal to) of the size of the cell image 12 is input to the extraction unit 85_1. The input data 101 having a size that is ½ of the size of the cell image 12 is input to the extraction unit 85_2. The input data 101 having a size that is ¼ of the size of the cell image 12 is input to the extraction unit 85_3. The input data 101 having a size that is ⅛ of the size of the cell image 12 is input to the extraction unit 85_4. In addition, the reason why the number of filters 102 is increased in the order of 32, 64, . . . from the extraction unit 85_1 to the extraction unit 85_4 is that, as the size of the input data 101 to be handled is reduced, the number of filters 102 is increased to extract various features included in the cell image 12.

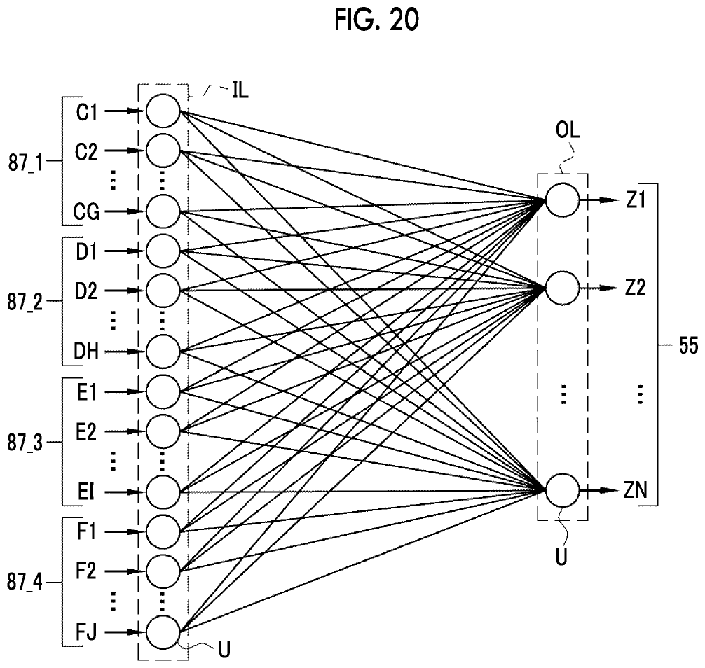

As illustrated in FIG. 20, the fully connected layer 95 has an input layer IL having units U that correspond to the number of target group feature amounts C, D, E, and F and an output layer OL including units U that correspond to the number of image feature amounts Z. Each unit U of the input layer IL and each unit U of the output layer OL are fully connected to each other, and weights are set for each unit. The target group feature amounts C, D, E, and F are input to each unit U of the input layer IL. The sum of the products of the target group feature amounts C, D, E, and F and the weights set for the units U is an output value from each unit U of the output layer OL. This output value is the image feature amount Z.

The restoration unit 77 is also provided with a fully connected unit, which is not illustrated. The fully connected unit of the restoration unit 77 converts the image feature amount Z of the image feature amount set 55 from the compression unit 76 into a target group feature amount corresponding to the target group feature amount F, contrary to the fully connected unit 86 of the compression unit 76. The restoration unit 77 gradually enlarges the target group feature amount map 87 generated in this way, contrary to the compression unit 76, and finally obtains the restored image 78. The restoration unit 77 performs a convolution process using the convolution layer in the process of gradually enlarging the target group feature amount map 87. This process is called an up-convolution process.

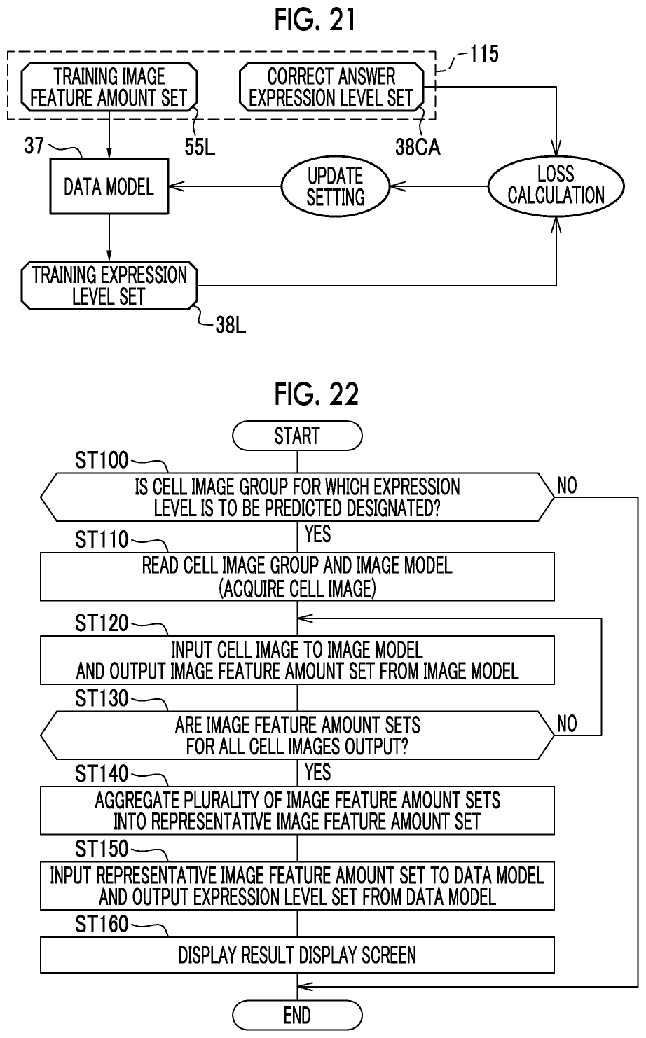

FIG. 21 illustrates an outline of a process of the data model 37 in the learning phase. In the learning phase, training data 115 is given to train the data model 37. The training data 115 is a set of a training image feature amount set 55L and a correct answer expression level set 38CA corresponding to the training image feature amount set 55L. The training image feature amount set 55L is obtained by inputting a certain cell image 12 to the image model 36. The correct answer expression level set 38CA is the result of actually measuring the expression levels of a plurality of types of ribonucleic acids X in the cell 13 included in the cell image 12 in a case in which the training image feature amount set 55L is obtained. In addition, examples of a method for measuring the expression level include quantitative polymerase chain reaction (Q-PCR), RNA sequencing, and single cell RNA sequencing.

In the learning phase, the training image feature amount set 55L is input to the data model 37. The data model 37 outputs a training expression level set 38L for the training image feature amount set 55L. The loss calculation of the data model 37 is performed on the basis of the training expression level set 38L and the correct answer expression level set 38CA. Then, the update setting of various coefficients of the data model 37 is performed according to the result of the loss calculation, and the data model 37 is updated according to the update setting.

In the learning phase of the data model 37, the series of processes of the input of the training image feature amount set 55L to the data model 37, the output of the training expression level set 38L from the data model 37, the loss calculation, the update setting, and the update of the data model 37 is repeated while the training data 115 is exchanged. The repetition of the series of processes ends in a case in which the prediction accuracy of the training expression level set 38L with respect to the correct answer expression level set 38CA reaches a predetermined set level. The data model 37 whose prediction accuracy has reached the set level is stored in the storage device 20 and is used by the second processing unit 48.

Next, the operation of the above-mentioned configuration will be described with reference to a flowchart illustrated in FIG. 22. First, in a case in which the operation program 30 is started in the cell culture evaluation device 10, as illustrated in FIG. 4, the CPU 22 of the cell culture evaluation device 10 functions as the RW control unit 45, the first processing unit 46, the aggregation unit 47, the second processing unit 48, and the display control unit 49.

In a case in which the cell image group 35 for which the expression level is to be predicted is designated on the designation screen 65 illustrated in FIG. 8 (YES in Step ST100), the RW control unit 45 reads the corresponding cell image group 35 and the image model 36 from the storage device 20 (Step ST110). The cell image group 35 and the image model 36 are output from the RW control unit 45 to the first processing unit 46. In addition, Step ST110 is an example of “acquisition” according to the technology of the present disclosure.

In the first processing unit 46, as illustrated in FIG. 5, one of the plurality of cell images 12 constituting the cell image group 35 is input to the image model 36. Then, the image feature amount set 55 is output from the image model 36 (Step ST120). The process in Step ST120 is continued until the image feature amount sets 55 are output for all of the plurality of cell images 12 constituting the cell image group 35 (NO in Step ST130). In addition, Step ST120 is an example of a “first process” according to the technology of the present disclosure.

In a case in which the image feature amount sets 55 are output for all of the plurality of cell images 12 constituting the cell image group 35 (YES in Step ST130), the image feature amount set group 56 composed of a plurality of image feature amount sets 55 is generated. The image feature amount set group 56 is output from the first processing unit 46 to the aggregation unit 47.

As illustrated in FIG. 6, the aggregation unit 47 aggregates the plurality of image feature amount sets 55 constituting the image feature amount set group 56 into one representative image feature amount set 55R that can be handled by the data model 37 (Step ST140). The representative image feature amount set 55R is output from the aggregation unit 47 to the second processing unit 48.

The data model 37 read from the storage device 20 by the RW control unit 45 is input to the second processing unit 48. In the second processing unit 48, the representative image feature amount set 55R is input to the data model 37 as illustrated in FIG. 7. Then, the expression level set 38 is output from the data model 37 (Step ST150). The expression level set 38 is output from the second processing unit 48 to the RW control unit 45. In addition, Step ST150 is an example of a “second process” according to the technology of the present disclosure.

The expression level set 38 is stored in the storage device 20 by the RW control unit 45. Further, the expression level set 38 is read from the storage device 20 by the RW control unit 45 and is then output to the display control unit 49.

Under the control of the display control unit 49, the result display screen 70 illustrated in FIG. 9 is displayed on the display 24 (Step ST160). The operator checks the expression level set 38 through the result display screen 70.

As described above, the CPU 22 of the cell culture evaluation device 10 functions as the RW control unit 45 as an acquisition unit, the first processing unit 46, and the second processing unit 48. The RW control unit 45 reads the cell image group 35 from the storage device 20 to acquire the cell images 12. The first processing unit 46 inputs the cell image 12 to the image model 36 and outputs the image feature amount set 55 composed of a plurality of types of image feature amounts Z related to the cell image 12 from the image model 36. The second processing unit 48 inputs the image feature amount set 55 (representative image feature amount set 55R) to the data model 37 and outputs the expression level set 38 composed of the expression levels of a plurality of types of ribonucleic acids X in the cell 13 from the data model 37.

The image feature amount Z is not obtained by inputting the cell image 12 to the commercially available image analysis software disclosed in JP2009-044974A, but is obtained by inputting the cell image 12 to the image model 36. Therefore, the image feature amount Z is not visually and intuitively understood by humans like the index value disclosed in JP2009-044974A, nor is arbitrarily set by humans. The image feature amount Z does not indicate a limited feature of the cell 13 like the index value disclosed in JP2009-044974A, but indicates a comprehensive feature of the cell 13. Therefore, it is possible to appropriately predict the expression levels of a plurality of types of ribonucleic acids X. As a result, it is possible to further accelerate the elucidation of the causal relationship between the expression level and the quality of the cell 13 and to greatly contribute to the improvement of the quality of the cell 13. In addition, it is possible to obtain expression level data non-invasively and at a low cost as compared to a method that actually measures the expression level such as Q-PCR.

The display control unit 49 performs control to display the expression level set 38. Therefore, it is possible to reliably inform the operator of the predicted expression level.

The RW control unit 45 reads the cell image group 35 from the storage device 20 to acquire a plurality of cell images 12 obtained by imaging one well 14, in which the cells 13 are cultured, a plurality of times. The first processing unit 46 inputs each of the plurality of cell images 12 to the image model 36 and outputs the image feature amount set 55 for each of the plurality of cell images 12 from the image model 36. Therefore, it is possible to output the expression level set 38 on the basis of the image feature amount set 55 output for each of the plurality of cell images 12 and to improve the reliability of the expression level set 38.

The aggregation unit 47 aggregates a plurality of image feature amount sets 55 output for each of the plurality of cell images 12 into one representative image feature amount set 55R that can be handled by the data model 37. The second processing unit 48 inputs the representative image feature amount set 55R to the data model 37, and the expression level set 38 is output from the data model 37. Therefore, the second processing unit 48 can reliably output the expression level set 38 from the data model 37.

As illustrated in FIG. 10, in the AE 75 having the compression unit 76 that converts the cell image 12 into the image feature amount set 55 and the restoration unit 77 that generates the restored image 78 of the cell image 12 from the image feature amount set 55, the compression unit 76 is used as the image model 36. Therefore, as illustrated in FIG. 11, the training data prepared in the learning phase is only the training cell image 12L. Therefore, it is possible to very easily obtain the image model 36 without requiring extra cost and time.

As illustrated in FIGS. 12 to 15, the compression unit 76 has a plurality of extraction units 85 and the fully connected unit 86. The plurality of extraction units 85 are prepared according to the size of an extraction target group in the cell image 12. The extraction units 85 extract the target group feature amount maps 87 composed of a plurality of types of target group feature amounts C, D, E, and F for the extraction target groups that each of the extraction units 85 is in charge of, using the convolution layers 90 and 91. The fully connected unit 86 converts a plurality of target group feature amount maps 87 output from the plurality of extraction units 85 into the image feature amount set 55, using the fully connected layer 95. Therefore, it is possible to obtain the target group feature amounts C, D, E, and F in a wide range from the target group feature amount map 87 showing the features of a relatively small extraction target group included in the cell image 12 to the target group feature amount map 87 showing the features of a relatively large extraction target group included in the cell image 12. As a result, it is possible to increase the comprehensiveness of the image feature amount Z.

In addition, FIG. 9 illustrates an example in which the expression level is displayed. However, the present disclosure is not limited thereto. A normalized expression level in a case in which the expression level of a reference ribonucleic acid X is 1 may be displayed. Further, a pie chart may be displayed instead of or in addition to the bar graph 71.

### Second Embodiment

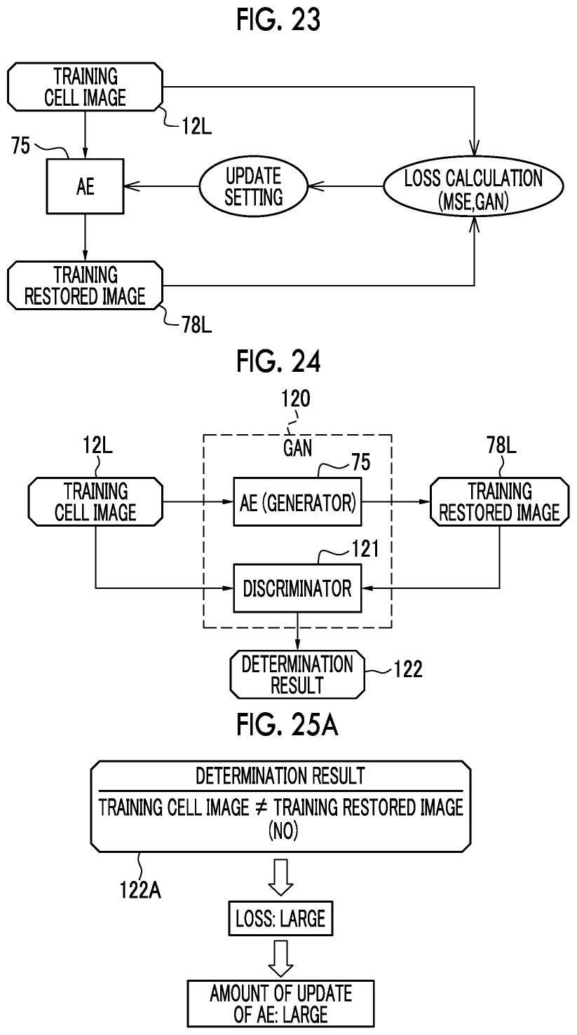

In a second embodiment illustrated in FIGS. 23 to 25, a generative adversarial network (hereinafter, abbreviated to GAN) 120 is used in the learning phase of the AE 75.

As illustrated in FIG. 23, in the second embodiment, the GAN 120 is used in the loss calculation of the AE 75 using a loss function such as a mean squared error. Specifically, as illustrated in FIG. 24, the AE 75 is incorporated as a generator of the GAN 120. Then, a discriminator 121 determines whether or not the training cell image 12L input to the AE 75 is the same as the training restored image 78L output from the AE 75. The discriminator 121 outputs a determination result 122.

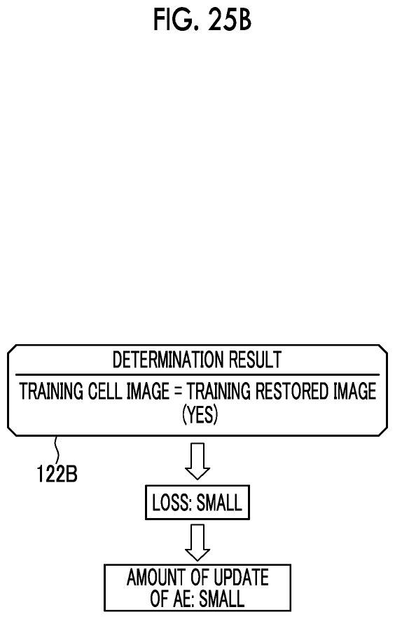

As illustrated in FIG. 25A, in a case in which the determination result 122A shows that the training cell image 12L is not the same as the training restored image 78L, the loss of AE 75 is estimated to be large. Therefore, in this case, the amount of update of the AE 75 is set to be relatively large. On the other hand, as illustrated in FIG. 25B, in a case in which the determination result 122B shows that the training cell image 12L is the same as the training restored image 78L, the loss of AE 75 is estimated to be small. Therefore, in this case, the amount of update of the AE 75 is set to be relatively small.

As described above, in the second embodiment, the AE 75 is trained using the GAN 120 having the discriminator 121 that determines whether or not the training cell image 12L is the same as the training restored image 78L. In a case in which only the loss function, such as the mean squared error, is used, the accuracy of restoration from the training cell image 12L to the training restored image 78L reaches a certain level. In contrast, in a case in which the GAN 120 is used, the accuracy of restoration can be further improved beyond the level of the accuracy of restoration that has reached the peak in a case in which only the loss function, such as the mean squared error, is used. As a result, it is possible to increase the reliability of the image feature amount Z and thus to increase the reliability of the expression level set 38.

### Third Embodiment

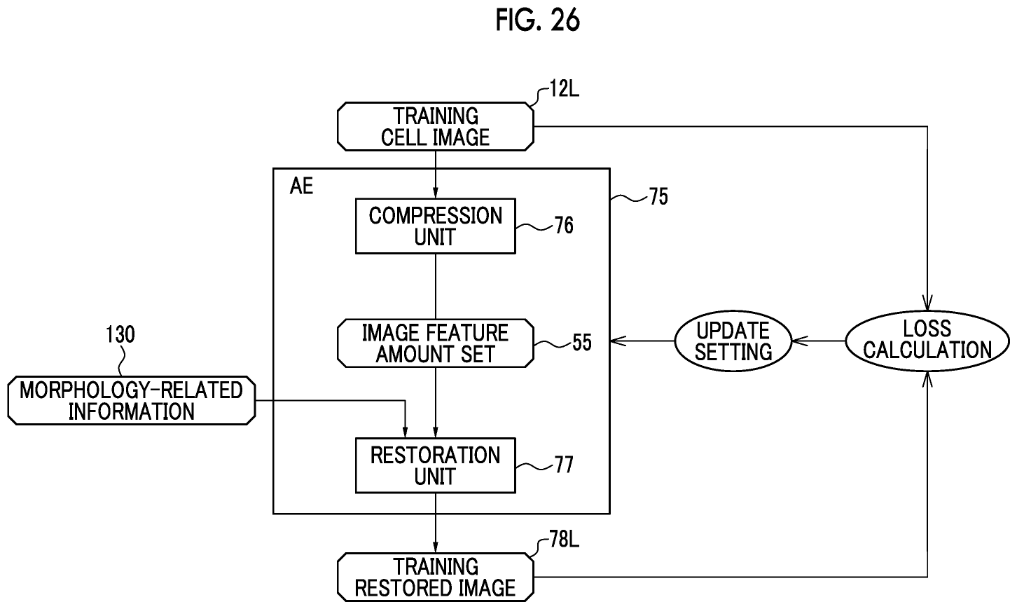

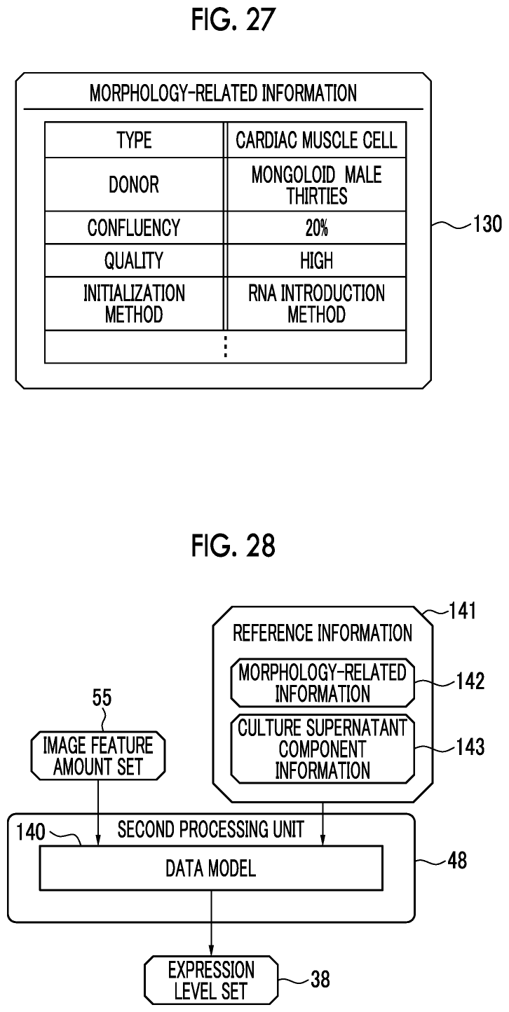

In a third embodiment illustrated in FIGS. 26 and 27, in addition to the image feature amount set 55 from the compression unit 76, morphology-related information 130 of the cell 13 is input to the restoration unit 77 to train the AE 75. That is, as illustrated in FIG. 26, in the third embodiment, in the learning phase of the AE 75, the morphology-related information 130 of the cell 13 included in the training cell image 12L is input to the restoration unit 77 together with the image feature amount set 55 from the compression unit 76. The restoration unit 77 restores the image feature amount set 55 to the training restored image 78L with reference to the morphology-related information 130.

As illustrated in FIG. 27, the morphology-related information 130 includes items such as the type, donor, confluency, quality, and initialization method of the cell 13. In addition to cardiac muscle cells given as an example, the types of the cells 13, such as nerve cells and hepatocytes, are registered in the item of the type. Alternatively, for example, hematopoietic stem cells, mesenchymal stem cells, skin stem cells, and progenitor cells may be registered in the item of the type. The race, gender, and age of the donor are registered in the item of the donor. A percentage indicating the proportion of a region in which the cell 13 is present to the entire region of the well 14 is registered in the item of the confluency. The quality level of the cell 13 determined by a well-known determination method is registered in the item of the quality. Specifically, in addition to a high level given as an example, a normal level and a low level are registered. A method used for the initialization (also called reprogramming) of the cell 13, such as an RNA introduction method given as an example, is registered in the item of the initialization method. In practice, numbers indicating these items are registered in each of the items of the type, donor, quality, and initialization method of the cell 13. Further, in addition to these items, an item, such as the number of culture days, is included in the morphology-related information 130.

As described above, in the third embodiment, the AE 75 is trained by inputting the morphology-related information 130 of the cell 13 to the restoration unit 77 in addition to the image feature amount set 55 from the compression unit 76. Therefore, the restoration unit 77 easily restores the image feature amount set 55 to the training restored image 78L, and it is possible to complete the training of the AE 75 in a short time.

The morphology-related information 130 includes the type, donor, confluency, quality, and initialization method of the cell 13. All of these items are important items that determine the morphology of the cell 13. Therefore, the restoration unit 77 easily restores the image feature amount set 55 to the training restored image 78L. In addition, the morphology-related information 130 may include at least one of the type, donor, confluency, quality, or initialization method of the cell.

### Fourth Embodiment

In a fourth embodiment illustrated in FIGS. 28 to 30, in addition to the image feature amount set 55, reference information 141 which is a reference for the output of the expression level set 38 is input to a data model 140.

In FIG. 28, the second processing unit 48 inputs the image feature amount set 55 and the reference information 141 to the data model 140. Then, the second processing unit 48 outputs the expression level set 38 from the data model 140. The reference information 141 includes morphology-related information 142 and culture supernatant component information 143. The morphology-related information 142 is information of the cell 13 included in the cell image 12 from which the image feature amount set 55 has been extracted. Similarly, the culture supernatant component information 143 is information of the cell 13 included in the cell image 12 from which the image feature amount set 55 has been extracted. The culture supernatant component information 143 is information related to a culture supernatant liquid collected from the well 14. The culture supernatant component information 143 is obtained by analyzing the culture supernatant liquid with an analyzer.

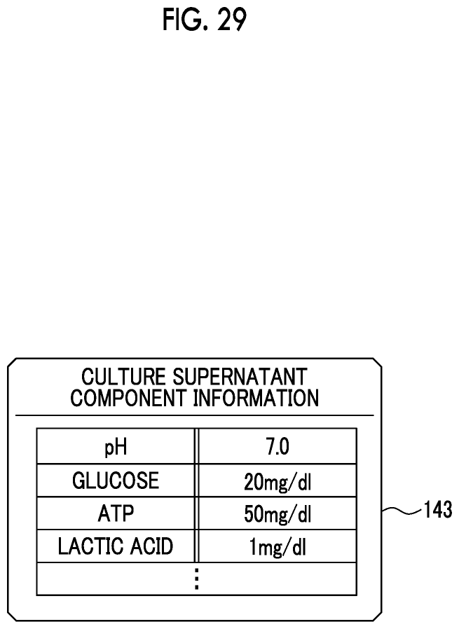

As illustrated in FIG. 29, the culture supernatant component information 143 has items such as potential of hydrogen (pH), glucose, adenosine triphosphate (ATP), and lactic acid. The amounts of these components are registered in the items of the glucose, the ATP, and the lactic acid. In addition, since the morphology-related information 142 is the same as the morphology-related information 130 according to the third embodiment, the illustration and description thereof will be omitted.

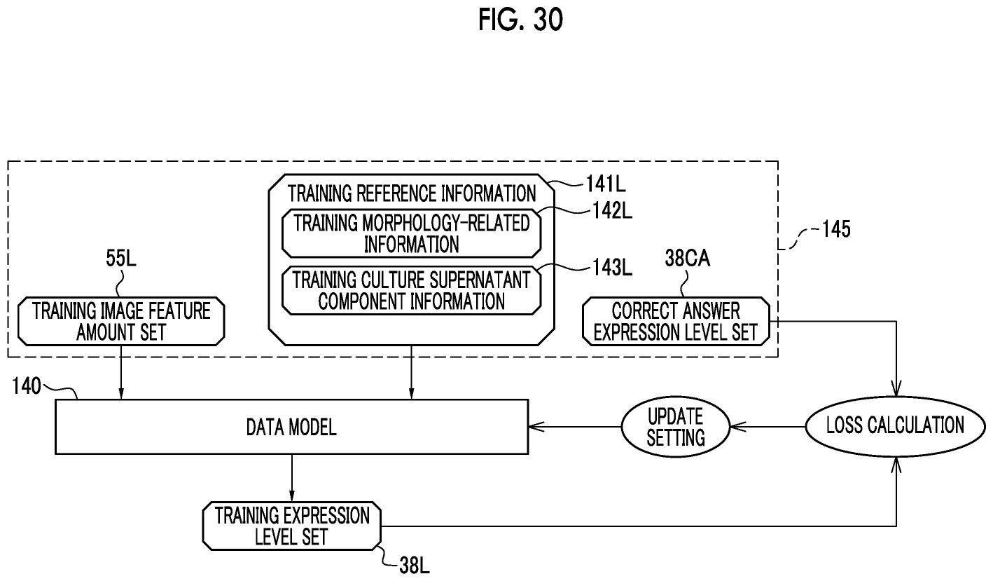

FIG. 30 illustrates an outline of a process in a learning phase of the data model 140. In the learning phase, training data 145 is given to train the data model 140. The training data 145 has training reference information 141L in addition to the training image feature amount set 55L and the correct answer expression level set 38CA illustrated in FIG. 21 of the first embodiment. The training reference information 141L includes training morphology-related information 142L and training culture supernatant component information 143L. Both the training morphology-related information 142L and the training culture supernatant component information 143L are information of the cell 13 included in the cell image 12 from which the training image feature amount set 55L has been extracted.

The training image feature amount set 55L and the training reference information 141L are input to the data model 140. The data model 140 outputs a training expression level set 38L for the training image feature amount set 55L and the training reference information 141L. Since the subsequent loss calculation and update setting processes are the same as those in the first embodiment, the description thereof will be omitted.

In the learning phase of the data model 140, the series of processes of the input of the training image feature amount set 55L and the training reference information 141L to the data model 140, the output of the training expression level set 38L from the data model 140, the loss calculation, the update setting, and the update of the data model 140 is repeated while the training data 145 is exchanged. The repetition of the series of processes ends in a case in which the prediction accuracy of the training expression level set 38L with respect to the correct answer expression level set 38CA reaches a predetermined set level. The data model 140 whose prediction accuracy has reached the set level is stored in the storage device 20 and is then used by the second processing unit 48.

As described above, in the fourth embodiment, the second processing unit 48 inputs the reference information 141, which is a reference for the output of the expression level set 38, to the data model 140 in addition to the image feature amount set 55. Therefore, it is possible to increase the prediction accuracy of the expression level set 38.

The reference information 141 includes the morphology-related information 142 of the cell 13 and the culture supernatant component information 143 of the cell 13. The morphology-related information 142 and the culture supernatant component information 143 are information that contributes to the prediction of the expression level set 38. Therefore, it is possible to increase the prediction accuracy of the expression level set 38.

The morphology-related information 142 includes the type, donor, confluency, quality, and initialization method of the cell 13. All of these items are important items that determine the morphology of the cell 13. Therefore, it is possible to increase the prediction accuracy of the expression level set 38. In addition, similarly to the morphology-related information 130, the morphology-related information 142 may include at least one of the type, donor, confluency, quality, or initialization method of the cell.

### Fifth Embodiment

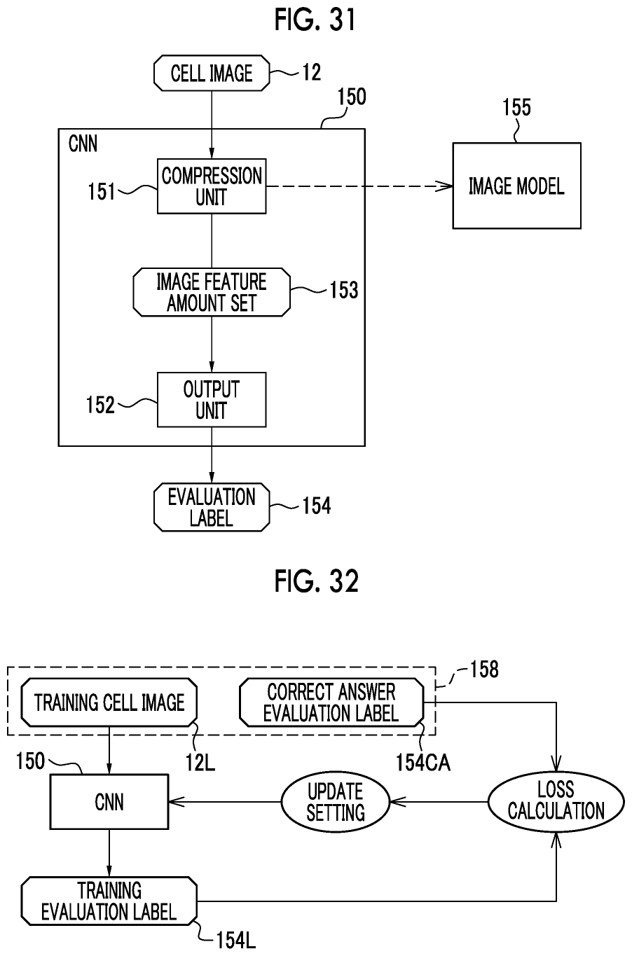

In a fifth embodiment illustrated in FIGS. 31 and 32, a compression unit 151 of a convolutional neural network (hereinafter, abbreviated to CNN) 150 is used as an image model 155, instead of the compression unit 76 of the AE 75.

As illustrated in FIG. 31, the CNN 150 has the compression unit 151 and an output unit 152. The cell image 12 is input to the compression unit 151. Similarly to the compression unit 76, the compression unit 151 converts the cell image 12 into an image feature amount set 153. The compression unit 151 transmits the image feature amount set 153 to the output unit 152. The output unit 152 outputs an evaluation label 154 for the cell 13 on the basis of the image feature amount set 153. The evaluation label 154 is, for example, the quality level of the cell 13 or the type of the cell 13. The compression unit 151 of the CNN 150 is used as the image model 155.

FIG. 32 illustrates an outline of a process in a learning phase of the CNN 150. In the learning phase, training data 158 is input to train the CNN 150. The training data 158 is a set of the training cell image 12L and a correct answer evaluation label 154CA corresponding to the training cell image 12L. The correct answer evaluation label 154CA is obtained by actually evaluating the cell 13 included in the training cell image 12L.

In the learning phase, the training cell image 12L is input to the CNN 150. The CNN 150 outputs a training evaluation label 154L for the training cell image 12L. The loss calculation of the CNN 150 is performed on the basis of the training evaluation label 154L and the correct answer evaluation label 154CA. Then, the update setting of various coefficients of the CNN 150 is performed according to the result of the loss calculation, and the CNN 150 is updated according to the update setting.

In the learning phase of the CNN 150, the series of processes of the input of the training cell image 12L to the CNN 150, the output of the training evaluation label 154L from the CNN 150, the loss calculation, the update setting, and the update of the CNN 150 is repeated while the training data 158 is exchanged. The repetition of the series of processes ends in a case in which the prediction accuracy of the training evaluation label 154L with respect to the correct answer evaluation label 154CA reaches a predetermined set level. The compression unit 151 of the CNN 150 whose prediction accuracy has reached the set level in this way is stored as the image model 155 in the storage device 20 and is then used by the first processing unit 46.

As described above, in the fifth embodiment, in the CNN 150 having the compression unit 151 that converts the cell image 12 into the image feature amount set 153 and the output unit 152 that outputs the evaluation label 154 for the cell 13 on the basis of the image feature amount set 153, the compression unit 151 is used as the image model 155. Therefore, in a case in which there is a sufficient amount of training data 158 which is a set of the training cell image 12L and the correct answer evaluation label 154CA, it is possible to create the image model 155 using the training data 158.

### Sixth Embodiment

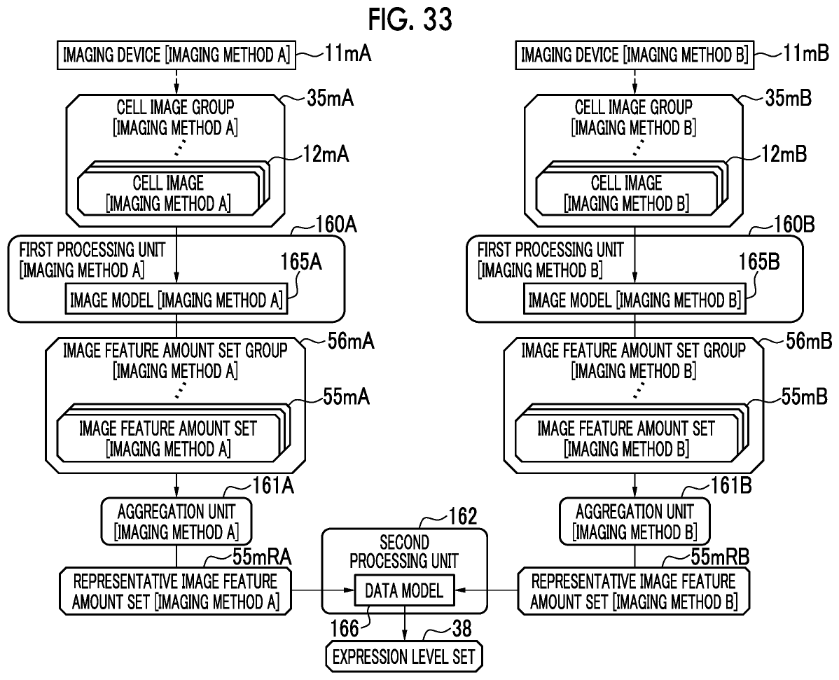

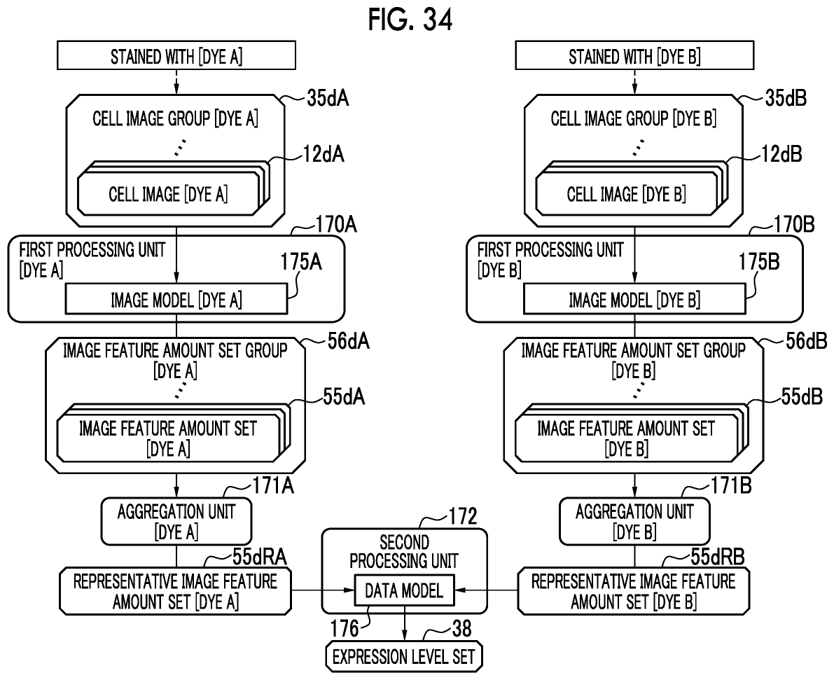

In the first embodiment, the cell images 12 obtained by imaging each of a plurality of regions 18 divided from the well 14 are given as an example of the plurality of cell images 12. However, the present disclosure is not limited thereto. As in a sixth embodiment illustrated in FIGS. 33 and 34, the plurality of cell images 12 may be cell images 12 captured by different imaging methods or cell images 12 obtained by imaging the cells 13 stained with different dyes.

FIG. 33 illustrates an example in which the plurality of cell images 12 are the cell images 12 captured by different imaging methods. Specifically, there are two types of cell images 12 of a cell image 12mA obtained by an imaging device 11mA using an imaging method A and a cell image 12mB obtained by an imaging device 11mB using an imaging method B. The imaging device 11mA is, for example, a bright-field microscope, and the imaging device 11mB is, for example, a phase-contrast microscope. In this case, a first processing unit 160A and an aggregation unit 161A for the cell image 12mA, a first processing unit 160B and an aggregation unit 161B for the cell image 12mB, and a second processing unit 162 are constructed in the CPU 22.

A cell image group 35mA and an image model 165A are input to the first processing unit 160A. The cell image group 35mA is composed of a plurality of cell images 12mA obtained by imaging each of the plurality of regions 18 using the imaging device 11mA. The first processing unit 160A inputs the cell image 12mA to the image model 165A and outputs an image feature amount set 55mA from the image model 165A. The first processing unit 160A outputs the image feature amount set 55mA for each of the plurality of cell images 12mA and outputs an image feature amount set group 56mA composed of a plurality of image feature amount sets 55mA to the aggregation unit 161A. The aggregation unit 161A aggregates the plurality of image feature amount sets 55mA into a representative image feature amount set 55mRA and outputs the representative image feature amount set 55mRA to the second processing unit 162. In addition, since the processes of the first processing unit 160B and the aggregation unit 161B are basically the same as the processes of the first processing unit 160A and the aggregation unit 161B except that “A” is changed to “B”, the description thereof will be omitted.

In addition to the representative image feature amount sets 55mRA and 55mRB, a data model 166 is input to the second processing unit 162. The second processing unit 162 inputs the representative image feature amount sets 55mRA and 55mRB to the data model 166 and outputs an expression level set 38 from the data model 166. In addition, the data model 166 is trained using the training image feature amount set (not illustrated) extracted from the cell image 12mA captured by the imaging device 11mA and the training image feature amount set (not illustrated) extracted from the cell image 12mB captured by the imaging device 11mB.

In a case in which the plurality of cell images 12 are the cell images 12 captured by different imaging methods, it is possible to further increase the prediction accuracy of the expression level set 38. This is because an object that clearly appears in the image may differ depending on the imaging method. For example, a phase object which is a colorless and transparent object does not appear in the cell image 12 captured by the bright-field microscope, but appears in the cell image 12 captured by the phase-contrast microscope. In other words, there are strengths and weaknesses depending on the imaging method. Therefore, the prediction accuracy of the expression level set 38 is further improved by comprehensively considering a plurality of imaging methods.

In addition, the bright-field microscope is given as an example of the imaging device 11mA, and the phase-contrast microscope is given as an example of the imaging device 11mB. However, the present disclosure is not limited thereto. For example, a dark-field microscope, a confocal microscope, a differential interference microscope, and a modulated contrast microscope may be used. Further, the different imaging methods are not limited to two types and may be three or more types. Furthermore, instead of using the image model 165 for each imaging method, an image model 165 common to a plurality of imaging methods may be used.

FIG. 34 illustrates an example in which the plurality of cell images 12 are cell images 12 obtained by imaging the cells 13 stained with different dyes. Specifically, there are a cell image 12dA obtained by imaging the cell 13 stained with a dye A and a cell image 12dB obtained by imaging the cell 13 stained with a dye B using the same imaging device 11 as that used for the dye A. The dye A is, for example, hematoxylin and eosin, and the dye B is, for example, crystal violet. In this case, a first processing unit 170A and an aggregation unit 171A for the cell image 12dA, a first processing unit 170B and an aggregation unit 171B for the cell image 12dB, and a second processing unit 172 are constructed in the CPU 22.

A cell image group 35dA and an image model 175A are input to the first processing unit 170A. The cell image group 35dA is composed of a plurality of cell images 12dA obtained by imaging the cell 13 stained with the dye A for each of a plurality of regions 18. The first processing unit 170A inputs the cell image 12dA to the image model 175A and outputs an image feature amount set 55dA from the image model 175A. The first processing unit 170A outputs the image feature amount set 55dA for each of the plurality of cell images 12dA and outputs an image feature amount set group 56dA composed of a plurality of image feature amount sets 55dA to the aggregation unit 171A. The aggregation unit 171A aggregates the plurality of image feature amount sets 55dA into a representative image feature amount set 55dRA and outputs the representative image feature amount set 55dRA to the second processing unit 172. In addition, since the processes of the first processing unit 170B and the aggregation unit 171B are basically the same as the processes of the first processing unit 170A and the aggregation unit 171B except that “A” is changed to “B”, the description thereof will be omitted.

In addition to the representative image feature amount sets 55dRA and 55dRB, a data model 176 is input to the second processing unit 172. The second processing unit 172 inputs the representative image feature amount sets 55dRA and 55dRB to the data model 176 and outputs an expression level set 38 from the data model 176. In addition, the data model 176 is trained with the training image feature amount set (not illustrated) extracted from the cell image 12dA obtained by imaging the cell 13 stained with the dye A and the training image feature amount set (not illustrated) extracted from the cell image 12dB obtained by imaging the cell 13 stained with the dye B.

In a case in which the plurality of cell images 12 are the cell images 12 obtained by imaging the cells 13 stained with different dyes, it is possible to further increase the prediction accuracy of the expression level set 38. As in the case illustrated in FIG. 33, this is because there are strengths and weaknesses depending on the dye. For example, some dyes stain cell nuclei, but some dyes stain sugar chains. Therefore, the prediction accuracy of the expression level set 38 is further improved by comprehensively considering a plurality of dyes.

In addition, the dyes are not limited to hematoxylin, eosin, and crystal violet. The dyes may be, for example, methylene blue, neutral red, and Nile blue. Further, the different dyes are not limited to two types and may be three or more types. Furthermore, instead of using the image model 175 for each dye, an image model 175 common to a plurality of dyes may be used.

For example, the aspect illustrated in FIG. 33 and the aspect illustrated in FIG. 34 may be combined with each other. For example, four types of cell images 12 obtained by imaging the cells 13 stained with the dyes A and B with the imaging devices 11A and 11B, respectively, may be used. Further, the plurality of cell images 12 may be cell images captured in time series such as on the first day and the second day of the culture.

In each of the above-described embodiments, a plurality of image feature amount sets 55 are aggregated into one representative image feature amount set 55R. However, the present disclosure is not limited thereto. The image feature amount sets may be aggregated into the number of representative image feature amount sets that can be handled by the data model 37. For example, 1000 image feature amount sets 55 may be aggregated into 10 representative image feature amount sets.

Instead of calculating the average values Z1AVE, Z2AVE, . . . , ZNAVE of the image feature amounts Z1, Z2, . . . , ZN, principal component analysis may be performed on each of the image feature amounts Z1, Z2, . . . , ZN to aggregate a plurality of image feature amount sets 55.

The aggregation unit 47 may not be provided. The expression level set 38 may be output for each of a plurality of image feature amount sets 55 extracted from a plurality of cell images 12. In this case, for example, the expression level sets 38 are output for a plurality of cell images 12 obtained by imaging each of the plurality of regions 18 divided from the well 14. Therefore, in the related art, one expression level set 38 is output for one well 14 due to the relationship between the measurement cost and the measurement time. However, the technology of the present disclosure can obtain a plurality of expression level sets 38 for one well 14. That is, it is possible to increase the resolution of the expression level set 38 for one well 14.

The hardware configuration of the computer constituting the cell culture evaluation device 10 can be modified in various ways. For example, the cell culture evaluation device 10 may be configured by a plurality of computers separated as hardware in order to improve processing capacity and reliability. For example, the functions of the RW control unit 45 and the display control unit 49 and the functions of the first processing unit 46, the aggregation unit 47, and the second processing unit 48 are distributed to two computers. In this case, the cell culture evaluation device 10 is configured by two computers.

As described above, the hardware configuration of the computer of the cell culture evaluation device 10 can be appropriately changed according to required performances, such as processing capacity, safety, and reliability. Further, not only the hardware but also an application program, such as the operation program 30, may be duplicated or may be dispersively stored in a plurality of storage devices in order to ensure safety and reliability.

In each of the above-described embodiments, for example, the following various processors can be used as the hardware configuration of processing units executing various processes, such as the RW control unit 45, the first processing unit 46, 160A, 160B, 170A, or 170B, the aggregation unit 47, 161A, 161B, 171A, or 171B, the second processing unit 48, 162, or 172, and the display control unit 49. The various processors include, for example, the CPU 22 which is a general-purpose processor executing software (operation program 30) to function as various processing units, a programmable logic device (PLD), such as a field programmable gate array (FPGA), which is a processor whose circuit configuration can be changed after manufacture, and/or a dedicated electric circuit, such as an application specific integrated circuit (ASIC), which is a processor having a dedicated circuit configuration designed to perform a specific process.

One processing unit may be configured by one of the various processors or a combination of two or more processors of the same type or different types (for example, a combination of a plurality of FPGAs and/or a combination of a CPU and an FPGA). In addition, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As described above, various processing units are configured by using one or more of the various processors as the hardware configuration.

In addition, specifically, an electric circuit (circuitry) obtained by combining circuit elements, such as semiconductor elements, can be used as the hardware configuration of the various processors.

In the technology of the present disclosure, the above-described various embodiments and/or various modification examples may be combined with each other. In addition, the present disclosure is not limited to each of the above-described embodiments, and various configurations can be used without departing from the gist of the present disclosure. Furthermore, the technology of the present disclosure extends to a storage medium that non-temporarily stores a program, in addition to the program.

The above descriptions and illustrations are detailed descriptions of portions related to the technology of the present disclosure and are merely examples of the technology of the present disclosure. For example, the above description of the configurations, functions, operations, and effects is the description of examples of the configurations, functions, operations, and effects of portions according to the technology of the present disclosure. Therefore, unnecessary portions may be deleted or new elements may be added or replaced in the above descriptions and illustrations without departing from the gist of the technology of the present disclosure. In addition, the description of, for example, common technical knowledge that does not need to be particularly described to enable the implementation of the technology of the present disclosure are omitted in order to avoid confusion and facilitate the understanding of portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case in which the connection of three or more matters is expressed by “and/or”.

All of the publications, the patent applications, and the technical standards described in the specification are incorporated by reference herein to the same extent as each individual document, each patent application, and each technical standard are specifically and individually stated to be incorporated by reference.

What is claimed is:

1. A cell culture evaluation device comprising at least one processor configured to:
acquire a cell image obtained by imaging a cell that is being cultured,
input the cell image to an image machine learning model to output an image feature amount set composed of a plurality of types of image feature amounts related to the cell image from the image machine learning model, and
input the image feature amount set to a data machine learning model to output an expression level set composed of expression levels of a plurality of types of ribonucleic acids of the cell from the data machine learning model.

2. The cell culture evaluation device according to claim 1,
wherein the processor is configured to perform control to display the expression level set.

3. The cell culture evaluation device according to claim 1,
wherein the processor is configured to:
acquire a plurality of the cell images obtained by imaging one culture container, in which the cell is cultured, a plurality of times, and
input each of the plurality of cell images to the image machine learning model to output the image feature amount set for each of the plurality of cell images from the image machine learning model.

4. The cell culture evaluation device according to claim 3,
wherein the processor is configured to:
aggregate a plurality of the image feature amount sets output for each of the plurality of cell images into a predetermined number of image feature amount sets that are capable of being handled by the data machine learning model, and
input the aggregated image feature amount sets to the data machine learning model to output the expression level set for each of the aggregated image feature amount sets from the data machine learning model.

5. The cell culture evaluation device according to claim 3,
wherein the plurality of cell images include at least one of cell images captured by different imaging methods, or cell images obtained by imaging the cells stained with different dyes.

6. The cell culture evaluation device according to claim 1,
wherein the processor is configured to input reference information, which is a reference for the output of the expression level set, to the data machine learning model, in addition to the image feature amount set.

7. The cell culture evaluation device according to claim 6,
wherein the reference information includes morphology-related information of the cell and culture supernatant component information of the cell.

8. The cell culture evaluation device according to claim 7,
wherein the morphology-related information includes at least one of a type, a donor, a confluency, a quality, or an initialization method of the cell.

9. The cell culture evaluation device according to claim 1,
wherein, the image machine learning model comprises a compression unit of an autoencoder, the autoencoder including the compression unit that converts the cell image into the image feature amount set, and a restoration unit that generates a restored image of the cell image from the image feature amount set.

10. The cell culture evaluation device according to claim 9,
wherein the compression unit includes:
a plurality of extraction units that are prepared according to a size of an extraction target group in the cell image, each of the plurality of extraction units extracting, using a convolution layer, a target group feature amount set composed of a plurality of types of target group feature amounts for the extraction target group corresponding to the each of the plurality of extraction unit, and
a fully connected unit that converts, using a fully connected layer, a plurality of the target group feature amount sets output from the plurality of extraction units into the image feature amount set.

11. The cell culture evaluation device according to claim 9,
wherein the autoencoder is trained using a generative adversarial network including a discriminator that determines whether or not the cell image is the same as the restored image.

12. The cell culture evaluation device according to claim 9,
wherein the autoencoder is trained by inputting morphology-related information of the cell to the restoration unit, in addition to the image feature amount set from the compression unit.

13. The cell culture evaluation device according to claim 12,
wherein the morphology-related information includes at least one of a type, a donor, a confluency, a quality, or an initialization method of the cell.

14. The cell culture evaluation device according to claim 1,
wherein the image machine learning model comprises a compression unit of a convolutional neural network, the convolutional neural network including the compression unit that converts the cell image into the image feature amount set, and an output unit that outputs an evaluation label for the cell on the basis of the image feature amount set.

15. A method for operating a cell culture evaluation device, the method being executed by a processor, the method comprising:
acquiring a cell image obtained by imaging a cell that is being cultured;
inputting the cell image to an image machine learning model to output an image feature amount set composed of a plurality of types of image feature amounts related to the cell image from the image machine learning model; and
inputting the image feature amount set to a data machine learning model to output an expression level set composed of expression levels of a plurality of types of ribonucleic acids of the cell from the data machine learning model.

16. A non-transitory storage medium storing a program that causes a computer to perform a cell culture evaluation processing, the cell culture evaluation processing comprising:
acquiring a cell image obtained by imaging a cell that is being cultured;
inputting the cell image to an image machine learning model to output an image feature amount set composed of a plurality of types of image feature amounts related to the cell image from the image machine learning model; and
inputting the image feature amount set to a data machine learning model to output an expression level set composed of expression levels of a plurality of types of ribonucleic acids of the cell from the data machine learning model.
